(19) United States
(12) Patent Application Publication
(10) Pub. No.: US 2024/0325384 A1
(43) Pub. Date: Oct. 3, 2024

(54) DOSING REGIMEN

(71) Applicant: AstraZeneca AB, Södertälje (SE)

(72) Inventors: Sabina Chiara COSULICH, Cambridge (GB); Adam DOWSON, Cambridge (GB); Jessica S. BROWN, Cambridge (GB); Edit Eva LUKACS, Cambridge (GB); Ganesh MOORTHY, Wilmington, DE (US); Chris WILKS, Cambridge (GB); Richard Edward FRANCIS, Cambridge (GB)

(21) Appl. No.: 18/618,221

(22) Filed: Mar. 27, 2024

Related U.S. Application Data
(60) Provisional application No. 63/493,320, filed on Mar. 31, 2023.

(51) Int. Cl. A61K 31/496; A61K 9/00; A61K 9/28; A61P 35/00

(52) U.S. Cl. CPC A61K 31/496; A61K 9/0053; A61K 9/28; A61P 35/00

(57) ABSTRACT

A method of treatment or prophylaxis of diseases or conditions in which inhibition of PARP1 is beneficial, comprising administering to the subject AZD5305 in a daily dose of 10 to 140 mg, wherein AZD5305 may be in the form of a pharmaceutically acceptable salt.

The present disclosure relates to methods of treatment or prophylaxis of diseases and conditions in which inhibition of PARP1 or PARP1 function is of therapeutic significance.

### BACKGROUND

PARP family of enzymes play an important role in a number of cellular processes, such as replication, recombination, chromatin remodeling, and DNA damage repair (O'Connor 2015).

Examples of PARP inhibitors and their mechanism of action are taught in e.g. WO2004/080976.

PARP1 and PARP2 are the most extensively studied PARPs for their role in DNA damage repair. PARP1 is activated by DNA damage breaks and functions to catalyse the addition of poly (ADP-ribose) (PAR) chains to target proteins. This post-translational modification, known as PARylation, mediates the recruitment of additional DNA repair factors to DNA lesions.

Following completion of this recruitment role, PARP auto-PARylation triggers the release of bound PARP from DNA to allow access to other DNA repair proteins to complete repair. Thus, the binding of PARP to damaged sites, its catalytic activity, and its eventual release from DNA are all important steps for a cancer cell to respond to DNA damage caused by chemotherapeutic agents and radiation therapy (Bai 2015).

Inhibition of PARP family enzymes has been exploited as a strategy to selectively kill cancer cells by inactivating complementary DNA repair pathways. A number of pre-clinical and clinical studies have demonstrated that tumour cells bearing deleterious alterations of BRCA1 or BRCA2, key tumour suppressor proteins involved in double-strand DNA break (DSB) repair by homologous recombination (HR), are selectively sensitive to small molecule inhibitors of the PARP family of DNA repair enzymes. Such tumours have deficient homologous recombination repair (HRR) pathways and are dependent on PARP enzymes function for survival. Although PARP inhibitor therapy has predominantly targeted BRCA-mutated cancers, PARP inhibitors have been tested clinically in non-BRCA-mutant tumors, those which exhibit homologous recombination deficiency (HRD) (Turner 2004).

It is believed that PARP inhibitors having improved selectivity for PARP1 may possess improved efficacy and reduced toxicity compared to other clinical PARP1/2 inhibitors. It is believed also that selective strong inhibition of PARP1 would lead to trapping of PARP1 on DNA, resulting in DNA double-strand breaks (DSBs) through collapse of replication forks in S-phase. It is believed also that PARP1-DNA trapping is an effective mechanism for selectively killing tumour cells having HRD.

“AZD5305” refers to a compound with the chemical name 5-{4-[(7-ethyl-6-oxo-5,6-dihydro-1,5-naphthyridin-3-yl)methyl]piperazin-1-yl}-N-methylpyridine-2-carboxamide and structure shown below:

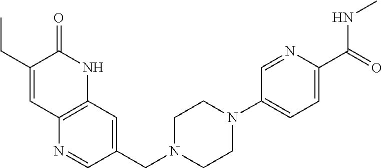 [SMILES: CCc1cc2ncc(CN3CCN(c4ccc(C(=O)NC)nc4)CC3)cc2[nH]c1=O]

AZD5305 is a potent and selective PARP1 inhibitor and PARP1-DNA trapper with excellent in vivo efficacy. AZD5305 is highly selective for PARP1 over other PARP family members, with good secondary pharmacology and physicochemical properties and excellent pharmacokinetics in preclinical species, and with reduced effects on human bone marrow progenitor cells in vitro.

The synthesis of AZD5305 is described in Johannes 2021 and in WO2021/013735, the contents of which are hereby incorporated by reference in their entirety.

To deliver AZD5305 to patients, safe and effective dosing regimens need to be developed with the aim of providing a more efficacious and less toxic cancer treatment compared with currently approved PARP inhibitors.

### SUMMARY

In some embodiments, disclosed is a method of treatment or prophylaxis of diseases or conditions in which inhibition of PARP1 is beneficial, comprising administering to the subject AZD5305 in a daily dose of 10 to 140 mg. AZD5305 may be administered as a pharmaceutically acceptable salt thereof, where the daily dosage is that of AZD5305 not in a salt form.

In some embodiments, disclosed is AZD5305, or a pharmaceutically acceptable salt thereof, for use in a method of treatment or prophylaxis of diseases or conditions in which inhibition of PARP1 is beneficial, wherein said AZD5305 is administered in a daily dose of 10 to 140 mg.

In some embodiments, disclosed is the use of AZD5305, or a pharmaceutically acceptable salt thereof, in the manufacture of a medicament for use in a method of treatment or prophylaxis of diseases or conditions in which inhibition of PARP1 is beneficial, wherein said treatment comprises the administration of said medicament comprising AZD5305 in a daily dose of 10 to 140 mg.

In some embodiments, disclosed is a pharmaceutical product comprising AZD5305 in a dose of 10 to 140 mg, where AZD5305 may be present as a pharmaceutically acceptable salt, and a pharmaceutically acceptable excipient, carrier or diluent.

In some embodiments, said disease or condition in which inhibition of PARP1 is beneficial is cancer. In some of these embodiments, the cancer is breast, ovary, pancreas, prostate, hematological, gastrointestinal such as gastric and colorectal, or lung cancer. In certain of these embodiments, the cancer is breast, ovary, pancreas or prostate cancer.

In some embodiments, the breast cancer is BRCA1m (BRCA1 mutated), BRCA2m (BRCA2 mutated), PALB2m (PALB2 mutated), RAD51Cm (RAD51C mutated), or RAD51Dm (RAD51D mutated) HER2-negative breast cancer.

In some embodiments, the ovarian cancer is BRCA1m (BRCA1 mutated), BRCA2m (BRCA2 mutated), PALB2m (PALB2 mutated), RAD51Cm (RAD51C mutated), or RAD51Dm (RAD51D mutated) ovarian cancer or ovarian cancer with high genomic instability.

In some embodiments, the prostate cancer is BRCA1/2 mutated prostate cancer or HRRm (HRR mutated) non-BRCA1/2 mutated prostate cancer.

### FIGURES

FIG. 1 is a flow chart of a clinical trial of AZD5305.

FIGS. 2A and 2B show the AZD5305 plasma concentration against the time after a single dose and after a dose during continuous dosing respectively.

FIG. 3 shows residual activity of PARP1 after a single dose of AZD5305.

FIGS. 4 to 7 show the response of 46 patients treated with AZD5305 as best percentage change in target lesion size by dose, tumour type, mutation type and dependent on prior PARPi treatment.

FIG. 8 shows the results of ctDNA measurements of 13 patients treated with AZD5305.

FIG. 9 shows the PSA₅₀ and ctDNA responses of a patient with prostate cancer treated with AZD5305.

FIGS. 10A and 10B are a baseline scan and second scan of the same patient treated with AZD5305 as in FIG. 9.

FIGS. 11A to 11D show the PSA₅₀ and ctDNA responses of four patients with prostate cancer treated with AZD5305.

FIG. 12 shows the CA15.3 response of a patient with breast cancer treated with AZD5305.

FIGS. 13A to 13C are baseline scans, first scans and second scans respectively of the same patient treated with AZD5305 as in FIG. 12.

FIG. 14 shows the CA125 response of a patient with ovarian cancer treated with AZD5305.

FIGS. 15A and 15B are baseline scans and third scans of the same patient treated with AZD5305 as in FIG. 14.

### DETAILED DESCRIPTION

In some embodiments, a free base AZD5305 is administered to a subject. In some embodiments, a pharmaceutically acceptable salt of AZD5305 is administered to a subject. In some embodiments, crystalline AZD5305 or a pharmaceutically acceptable salt of AZD5305 is administered to a subject.

The language “pharmaceutical composition” includes compositions comprising an active ingredient and a pharmaceutically acceptable excipient, carrier or diluent, wherein the active ingredient is AZD5305 or a pharmaceutically acceptable salt thereof. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. In some embodiments, the pharmaceutical compositions are in solid dosage forms, such as capsules, tablets, granules, powders or sachets. In some embodiments, the pharmaceutical compositions are in the form of a sterile injectable solution in one or more aqueous or non-aqueous non-toxic parenterally acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The language “treat,” “treating” and “treatment” includes the reduction or inhibition of enzyme or protein activity related to PARP-1 in a subject, amelioration of one or more symptoms of diseases or conditions in which inhibition of PARP1 is beneficial in a subject, or the slowing or delaying of progression of diseases or conditions in which inhibition of PARP1 is beneficial in a subject. The language “treat,” “treating” and “treatment” also includes the reduction or inhibition of the growth of a tumor or proliferation of cancerous cells in a subject.

The language “inhibit”, “inhibition” or “inhibiting” includes a decrease in the baseline activity of a biological activity or process.

The term “subject” includes warm-blooded mammals, for example, primates, dogs, cats, rabbits, rats, and mice. In some embodiments, the subject is a primate, for example, a human. In some embodiments, the subject is suffering from diseases or conditions in which inhibition of PARP1 is beneficial.

In some embodiments, AZD5305 or a pharmaceutically acceptable salt thereof are administered in a treatment cycle. In some embodiments, AZD5305 or a pharmaceutically acceptable salt thereof is continuously administered in the treatment cycle.

The term “continuous” or “continuously” refers to administration of a therapeutic agent, e.g. AZD5305, at regular intervals without stopping or interruption, i.e., no void day. By “void day”, it is meant a day when a therapeutic agent is not administered.

A “cycle”, “treatment cycle” or “dosing schedule”, as used herein, refers to a period of treatment that is repeated on a regular schedule. For example, the treatment can be given for one week, two weeks, or three weeks wherein AZD5305 is administered. In some embodiments, a treatment cycle is about 1 week to about 3 months. In some embodiments, a treatment cycle is about 5 days to about 1 month. In some embodiments, a treatment cycle is about 1 week to about 3 weeks. In some embodiments, a treatment cycle is about 1 week, about 10 days, about 2 weeks, about 3 weeks, about 4 weeks, about 2 months, or about 3 months.

In some embodiments, AZD5305 or a pharmaceutically acceptable salt thereof is administered to the human subject in one or more treatment cycles, e.g., a treatment course. A “treatment course” comprises multiple treatment cycles, which can be repeated on a regular schedule, or adjusted as a tapered schedule as the patient's disease progression is monitored. For example, a patient's treatment cycles can have longer periods of treatment and/or shorter periods of rest at the beginning of a treatment course (e.g., when the patient is first diagnosed), and as the cancer enters remission, the rest period lengthens, thereby increasing the length of one treatment cycle. The period of time for treatment and rest in a treatment cycle, the number of treatment cycles, and the length of time for the treatment course can be determined and adjusted throughout the treatment course by the skilled artisan based on the patient's disease progression, treatment tolerance, and prognosis. In some embodiments, the method comprises 1 to 10 treatment cycles. In some embodiments, the method comprises 2 to 8 treatment cycles.

In some embodiments, AZD5305 or a pharmaceutically acceptable salt thereof is administered for 28 days in a 28-day treatment cycle.

In some embodiments, AZD5305 or a pharmaceutically acceptable salt thereof is administered orally. In some embodiments, AZD5305 or a pharmaceutically acceptable salt thereof is in tablet dosage form.

In some embodiments, AZD5305 is administered in a dose of 10 to 140 mg per day. The daily dose may be up to 20 mg, 30 mg, 40 mg, 50 mg, 60 mg, 70 mg, 80 mg, 90 mg, 100 mg, 110 mg, 120 mg, 130 mg or 140 mg. The daily dose may be at least 10 mg, 20 mg, 30 mg, 40 mg, 50 mg, 60 mg, 70 mg, 80 mg, 90 mg, 100 mg, 110 mg, 120 mg or 130 mg.

In some embodiments, AZD5305 is administered in a daily dose of 10 mg, 20 mg, 30 mg, 40 mg, 50 mg, 60 mg, 70 mg, 80 mg, 90 mg, 100 mg, 110 mg, 120 mg, 130 mg or 140 mg.

In some embodiments, AZD5305 is administered once a day (QD). In some embodiments, AZD5305 is administered in a dose of 10 to 140 mg QD. The once daily dose may be up to 20 mg, 30 mg, 40 mg, 50 mg, 60 mg, 70 mg, 80 mg, 90 mg, 100 mg, 110 mg, 120 mg, 130 mg or 130 mg. The once daily dose may be at least 10 mg, 20 mg, 30 mg, 40 mg, 50 mg, 60 mg, 70 mg, 80 mg, 90 mg, 100 mg, 110 mg, 120 mg or 130 mg.

In some embodiments, AZD5305 is administered in a once daily dose (QD) of 10 mg, 20 mg, 30 mg, 40 mg, 50 mg, 60 mg, 70 mg, 80 mg, 90 mg, 100 mg, 110 mg, 120 mg, 130 mg or 140 mg.

In some embodiments, the cancer treated may be deficient in Homologous Recombination (HR) dependent DNA DSB repair activity. The HR dependent DNA DSB repair pathway repairs double-strand breaks (DSBs) in DNA via homologous mechanisms to reform a continuous DNA helix (Khanna and Jackson 2001). The components of the HR dependent DNA DSB repair pathway include, but are not limited to, ATM (NM_000051), RAD51 (NM_002875), RAD51L1 (NM_002877), RAD51C (NM_002876), RAD51L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE11A (NM_005590) and NBS1 (NM_002485). Other proteins involved in the HR dependent DNA DSB repair pathway include regulatory factors such as EMSY (Hughes-Davies 2003). HR components are also described in Wood 2001.

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood 2001) and include the components listed above.

In some embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies 2003).

BRCA1 and BRCA2 are known tumour suppressors whose wild-type alleles are frequently lost in tumours of heterozygous carriers (Jasin 2002; Tutt 2002). The association of BRCA1 and/or BRCA2 mutations with breast cancer is well-characterised in the art (Radice 2002). Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast, ovary, pancreas, prostate, hematological, gastrointestinal and lung cancer.

In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen and Ostrander 1992; Chappuis and Foulkes 2002; Janatová 2003; Jancárková 2003). Determination of amplification of the BRCA2 binding factor EMSY is described in Hughes-Davies 2003.

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

In some embodiments, AZD5305 is administered as a monotherapy.

In some embodiments, AZD5305 is administered in combination with one or more other agents which can be used in treating diseases and conditions in which inhibition of PARP1 or PARP 1 function is of therapeutic significance.

### Preclinical Studies

Preclinical characterization of AZD5305 is reported in Illuzzi 2022.

AZD5305 was tested in vitro for PARylation inhibition, PARP-DNA trapping, and antiproliferative abilities. In vivo efficacy was determined in mouse xenograft and PDX models. The potential for hematologic toxicity was evaluated in rat models, as monotherapy and combination. It was reported that AZD5305 was a highly potent and selective inhibitor of PARP1 with 500-fold selectivity for PARP1 over PARP2. AZD5305 inhibits growth in cells with deficiencies in DNA repair, with minimal/no effects in other cells. Unlike first-generation PARPi, AZD5305 has minimal effects on hematologic parameters in a rat pre-clinical model at predicted clinically efficacious exposures. Animal models treated with AZD5305 at doses ≥0.1 mg/kg once daily achieved greater depth of tumor regression compared to olaparib 100 mg/kg once daily, and longer duration of response.

### EXAMPLES

The compounds of the application will now be further explained by reference to the following non-limiting examples.

### Example 1—Clinical Trial

This study plans to investigate the safety and preliminary clinical efficacy of AZD5305 in patients with advanced/metastatic cancer and HRD.

Part A is a dose-escalation study and will include patients with advanced/relapsed ovarian cancer, HER2 (human epidermal growth factor receptor 2)-negative breast cancer, CRPC (castration-resistant prostate cancer) or pancreatic cancer. All patients should have a loss of function or predicted loss of function mutation in BRCA1, BRCA2, PALB2, RAD51C or RAD51D.

Part B will have expansion cohorts in specific indications:
Cohort B1—Advanced/relapsed HER2-negative breast cancer patients with BRCA1m, BRCA2m, PALB2m, RAD51Cm or RAD51Dm
Cohort B2—PSR (platinum sensitive relapsed) high grade epithelial ovarian cancer patients with loss of function mutations in BRCA1, BRCA2, PALB2, RAD51C or RAD51D.
Cohort B3—PSR high grade epithelial ovarian cancer patients with high levels of genomic instability (and absence of loss of function mutations in BRCA1, BRCA2, PALB2, RAD51C or RAD51D)
Cohort B4—Histologically or cytologically confirmed advanced/metastatic CRPC and a qualifying loss of function mutation in BRCA1 or BRCA2.
Cohort B5—Histologically or cytologically confirmed advanced/metastatic CRPC and a qualifying loss of function alteration (in HRRm (ATM, BARD1, BRIP1, CDK12, CHEK1, CHEK2, FANCL, PALB2, RAD51B, RAD51C, RAD51D and RAD54L [without BRCA1/2m]).

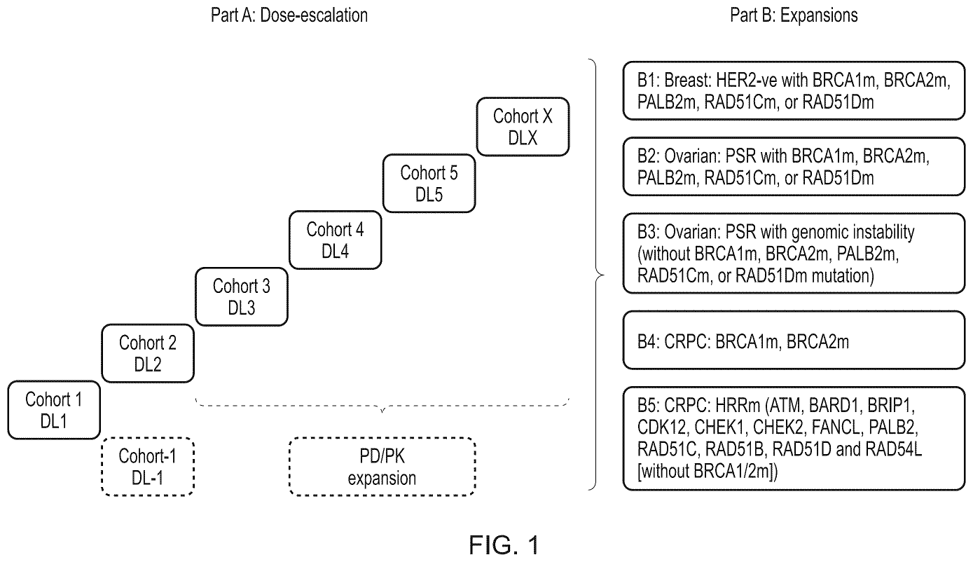

For patients in Part A, up to one prior line of treatment with PARP inhibitor is allowed (either in the metastatic or treatment setting). For Part B, patients must be PARP inhibitor naïve. The study flow chart is shown in FIG. 1 (DL=dose level).

### Objectives and Endpoints

TABLE 1
Primary Objectives and Endpoints

| Primary/Safety Objective | Primary Endpoints/Variables |
|---|---|
| To assess the safety and tolerability | Incidence of Adverse Events |
| of AZD5305 as monotherapy and in | (AEs)/Serious Adverse Events |
| combination with anti-cancer agents | (SAEs) |
| in patients with advanced | Dose-Limiting Toxicities (DLTs) |
| malignancies | Maximum Tolerated Dose (MTD) |
| | Changes from baseline in |
| | laboratory findings, physical |
| | examination, ECOG PS (Eastern |
| | Cooperative Oncology Group |
| | Performance Status), |
| | Electrocardiograms (ECGs) and |
| | vital signs |

TABLE 2
Secondary Objectives and Endpoints

| Secondary Objective | Endpoints/Variables |
|---|---|
| To characterize the | Plasma concentrations of AZD5305 |
| Pharmacokinetics (PK) of | and plasma PK parameters, |
| AZD5305 in plasma, | including but not limited to Area |
| following a single dose and | Under Curve (AUC), Cₘₐₓ |
| at steady state after multiple | (maximum plasma concentration of |
| dosing, when given orally | the drug) and Tₘₐₓ (the time taken |
| | to reach the maximum |
| | concentration) as data allow, of |
| | AZD5305 after single dose and |
| | multiple doses administration |
| To assess the preliminary | Radiological response evaluated |
| anti-tumour activity of | according to response evaluation |
| AZD5305 | criteria in solid tumours (RECIST |
| | v1.1; Response Evaluation Criteria |
| | In Solid Tumours version 1.1) |
| | Best percentage change in |
| | target lesion |
| | ORR (objective response rate), |
| | DoR (duration of response), |
| | PFS (progression-free |
| | survival), TTR (time to |
| | response) |
| | For ovarian cancer patients, CA125 |
| | (Cancer antigen 125) response |
| | evaluated according to the GCIG |
| | (Gynecologic Cancer InterGroup) |
| | criteria |
| | Specifically, for prostate cancer |
| | patients (in addition to what is in |
| | the core protocol): |
| | ORR and rPFS using RECIST |
| | 1.1 (soft tissue) and PCWG3 |
| | (bone) criteria |
| | Proportion of subjects |
| | achieving a ≥50% decrease in |
| | PSA from baseline to the |
| | lowest post-baseline PSA |
| | result, confirmed by a second |
| | consecutive PSA assessment |
| | at least 3 weeks later (PSA₅₀ |
| | response) |
| To evaluate pharmacodynamics | Includes, but is not limited to |
| (PD) of AZD5305 in tumour | assessment pH2AX - Ser139 |
| tissue when given orally as | (phospho-histone 2AX - Serine |
| monotherapy | 139) PD biomarker modulations at |
| | baseline visit 1 and during |
| | treatment. |

TABLE 3
Exploratory Objectives and Endpoints

| Exploratory Objective | Endpoints/Variables |
|---|---|
| To investigate the presence and/or | Metabolite identification |
| identify (in plasma and urine) drug | |
| metabolites of AZD5305 and, if | |
| appropriate, characterise PK | |
| To understand the CYP3A4 induction | Pre-dose to post-dose 4-β- |
| potential of AZD5305 | hydroxy cholesterol ratio |
| To explore relationship between PK | Correlation of PK with other |
| concentration or parameters and | primary/secondary/exploratory |
| selected endpoints (which may | endpoints in patients treated with |
| include PD parameters, efficacy, | AZD5305. |
| and/or safety), where deemed | |
| appropriate | |
| To characterise the PD of AZD5305, | Assessment of modulation from |
| following a single dose and at steady | baseline (Visit 1) or pre- |
| state after multiple dosing, when | treatment in PD biomarkers from |
| given orally | PBMC (peripheral blood |
| | mononuclear cell), and/or |
| | tumoural samples |
| | Analysis of PARP1 inhibition |
| | including, but not limited to PAR |
| | (poly adenosine diphosphate- |
| | ribose), PARP-DNA trapping and |
| | pH2AX (Ser139) |
| To investigate predictive markers of | Assessment of association of |
| response and acquired resistance to | biomarkers including but not |
| AZD5305 that may be observed in | limited to gene mutations, |
| tumour and ctDNA (circulating | genomic alterations, mRNA, |
| tumour DNA) from patients treated | proteins, RAD51 foci with |
| with AZD5305 | observed response or resistance |
| | to AZD5305 |
| To determine the efficacy of | Clinical efficacy associated with |
| AZD5305 according to biomarker | biomarkers including but not |
| subgroups defined from the blood, | limited to: |
| tumour or ctDNA | tBRCAm (tumour BRCA |
| | mutation), gBRCAm (germline |
| | BRCA mutation), sBRCAm |
| | (somatic BRCA mutation) |
| | BRCA methylation, non- |
| | BRCAm (patients without |
| | BRCA or HRR mutations |
| | HRRm - individually and as a |
| | panel) |
| | Genomic instability status |
| | RAD51 foci |
| To explore the impact of the study | Correlation of percentage change |
| treatment on ctDNA levels in patients | in ctDNA mutation frequency |
| by exploring the relationship between | with PFS and other clinical and |
| ctDNA kinetics and clinical response/ | translational endpoints |
| progression, clonal evolution of | |
| disease, predictive biomarkers of | |
| response and resistance | |
| To collect and store germline DNA to | Presence or absence of CHIP |
| assess CHIP (Clonal Haematopoiesis | mutations |
| of Indeterminate Potential) to aid | Germline DNA may also be used |
| interpretation of ctDNA mutation | for possible exploration of |
| monitoring | genetic factors associated with |
| | drug response and tolerability |
| To collect and store plasma, serum | This may include analysis of |
| blood or tissue samples for | tumour specific and circulating |
| companion diagnostics development | biomarkers, such as tumour |
| and exploratory analysis | DNA, genes/genetic variations, |
| | mRNA, proteins, cytokines or |
| | metabolites. |
| | Exploratory biomarkers |
| | Pharmacogenomics (genomics |
| | initiative) |
| To assess the overall survival when | Overall Survival (OS) |
| treated by AZD5305 | |
| To assess the preliminary anti-tumour | Proportion of subjects achieving |
| activity of AZD5305 | a decline in the number of CTCs |
| | from ≥5 cells/7.5mL to |
| | <5 cells/7.5 mL blood (CTC |
| | conversion rate) |

### Number of Patients

Part A will require approximately 45 patients (n=3 to 6 per cohort). This number of patients is expected to provide a model-based estimate of MTD with a target toxicity of 25%. Doses and/or schedules of AZD5305 will be defined by taking the Bayesian adaptive design scheme and PK into consideration. Dose-escalation will stop at the MTD, MFD, or the RP2D.

Part A, dose-escalation cohorts may be expanded to evaluate further the safety, tolerability, PK, PD, and biological activity of AZD5305, provided the dose has already been declared as tolerated. Patients with specific tumour types and prior treatment status (within the current eligibility criteria) may be enrolled in these Part A expansion cohorts based on emerging data. Dose-escalation will continue in parallel. Patients recruited for tumour PD analysis will be required to have paired biopsies.

In Part B, expansion cohorts may be initiated to investigate preliminary efficacy and build on safety data in specific patient populations. Expansions may be initiated at more than one dose level depending on emerging data. A sample size of approximately 30 evaluable patients per Part B expansion cohort is sufficient for preliminary detection of an efficacy signal. A mean of 2 cohorts for each of the 4 patient populations in B1-B4 is estimated plus 1 cohort for B5. Thus, in total, approximately 270 patients are planned.

These are summarised in the study flow chart shown in FIG. 1.

Each patient must meet all of the inclusion criteria and none of the exclusion criteria for this study at the time of starting study treatment. Under no circumstances can there be exceptions to this rule.

### Core Inclusion Criteria

For inclusion in the study, patients must fulfil all the following criteria:
1. Provision of signed and dated, written informed consent prior to any study specific procedures, sampling and analyses. If a patient declines to participate in any voluntary exploratory research and/or optional genetic component of the study, there will be no penalty or loss of benefit to the patient and he/she will not be excluded from other aspects of the study.
2. Age ≥18 at the time of screening
3. Patients must have histological or cytological confirmation of advanced malignancy considered to be suitable for study treatment.
4. Eastern Cooperative Oncology Group Performance status (ECOG PS: 0-2) with no deterioration over the previous 2 weeks
5. Life expectancy ≥12 weeks
6. Progressive cancer at the time of study entry
7. Patients must have evaluable disease as defined in module-specific criteria for Part A and Part B
8. Female subjects of childbearing potential:
   (a) Must have a negative pregnancy test result at screening and prior to each cycle of study treatment
   (b) If sexually active with a non-sterilized male partner, must use at least one highly effective method of birth control from screening to approximately 6 months after the last dose of study treatment in combination with one effective method (male condom plus spermicide) from screening until approximately 6 months after the last dose of study treatment.
9. Female subjects must not breastfeed and must not donate or retrieve ova for their own use from screening to approximately 6 months after the last dose of study treatment.
10. Non-sterilized male patients who are sexually active with a female partner of childbearing potential must use a condom with spermicide from screening to approximately 6 months after the last dose of study treatment (Note: Male condoms are not reliable as a sole contraception method. In countries where spermicide is not approved, use of male condoms without spermicide is permitted). It is strongly recommended that female partners of male patients also use at least one highly effective method of contraception throughout this period. In addition, male patients must refrain from fathering a child or donating sperm during the study and for approximately 6 months after the last dose of study treatment.
11. Adequate organ and marrow function (in the absence of transfusions or growth factor support within 14 days prior to enrolment) as defined below in Table 4:

TABLE 4
Criteria for adequate organ and marrow function

| Category | Parameter | Value |
|---|---|---|
| Hematological | Hemoglobin | ≥9.0 g/dl (5.59 mmol/L) |
| | Absolute neutrophil | ≥1.5 × 10⁹/L |
| | count | (1,500 per mm³) |
| | Platelet count | ≥100 × 10⁹/L |
| | | (100,000 per mm³) |
| | INR | ≤1.5 |
| Hepatic | Total bilirubin | ≤1.5 × ULN in the absence |
| | | Gilbert's syndrome |
| | | ≤3 × ULN if the subject has |
| | | Gilbert's syndrome |
| | ALT and AST | ≤2.5 × ULN in the absence |
| | | of liver metastases |
| | | ≤5 × ULN in presence of |
| | | liver metastases ª |
| Renal | Calculated creatinine | ≥45 mL/minute |
| | clearance by modified | |
| | Cockcroft-Gault | |
| | (Rostoker 2007) | |

ALT = alanine transaminase;
AST = aspartate transaminase;
INR = international normalized ratio;
ULN = upper limit normal
^{a} In the presence of liver metastases and raised ALT/AST between 2.5-5 × ULN, patients can only be enrolled if total bilirubin level is <1.5 × ULN.

12. For Part B expansion cohorts: Provision of formalin-fixed and paraffin embedded (FFPE) tumour specimen is mandatory, where available, except if stated that it is optional in a specific Module. An archival tissue specimen is preferred but a new tissue sample may be used (if, for example, it is routine at a site to undertake biopsy of metastatic lesions).

### Core Exclusion Criteria

Patients must not enter the study if any of the following exclusion criteria are fulfilled:
1. Treatment with any of the following:
   (a) Nitrosourea or mitomycin C within 6 weeks of the first dose of study treatment
   (b) Any investigational agents or study drugs from a previous clinical study within 5 half-lives or 3 weeks (whichever is shorter) of the first dose of study treatment
   (c) Any other anti-cancer treatment within the following time periods prior to the first dose of study treatment:
      (i) Cytotoxic and non-cytotoxic treatment: 3 weeks or 5 half-lives (whichever is shorter)
      (ii) Biological products including immuno-oncology agents: 4 weeks before enrolment
   (d) Any live virus or bacterial vaccine within 28 days of the first dose of study treatment
2. Concomitant use of medications or herbal supplements known to be cytochrome P450 3A4 (CYP3A4) strong and moderate inhibitors or inducers, or inability to stop use at least 21 days or at least 5 half-lives (whichever is shorter) before the first dose of study treatment until 30 days after the last dose of study treatment.
3. Concomitant use of drugs that are known to prolong or shorten QT and have a known risk of Torsades de Pointes.
4. During the 4 weeks prior to the first dose, receiving continuous corticosteroids at a dose of >10 mg prednisone/day or equivalent for any reason.
5. Major surgery within 4 weeks of the first dose of study treatment.
6. Radiotherapy with a wide field of radiation within 4 weeks or radiotherapy with a limited field of radiation for palliation within 2 weeks of the first dose of study treatment.
7. With the exception of alopecia, any unresolved toxicities from prior therapy greater than Common Terminology Criteria for Adverse Events (CTCAE) Grade 1 at the time of starting study treatment.
8. Any history of persisting (>2 weeks) severe pancytopenia due to any cause (absolute neutrophil count [ANC] <0.5×10⁹/L or platelets <50×10⁹/L)
9. Spinal cord compression or brain metastases unless asymptomatic, treated and stable and not requiring continuous corticosteroids at a dose of >10 mg prednisone/day or equivalent for at least 4 weeks prior to start of study treatment
10. As judged by the Investigator, any evidence of severe or uncontrolled systemic diseases, including, active bleeding diatheses, or active infection including hepatitis B, hepatitis C and human immunodeficiency virus (HIV). Screening for chronic conditions is not required.
11. Patients with any known predisposition to bleeding (e.g., active peptic ulceration, recent [within 6 months] haemorrhagic stroke, proliferative diabetic retinopathy).
12. Any of the following cardiac criteria:
   (a) Mean resting corrected QT interval (QTcF)>450 milliseconds or QTcF<340 milliseconds obtained from triplicate electrocardiograms (ECGs) and averaged, recorded within 5 minutes
   (b) Any factors that increase the risk of QT prolongation, shortening or risk of arrhythmic events such as hypokalaemia, congenital long or short QT syndrome, family history of long QT syndrome, familial short QT syndrome or unexplained sudden death under 40 years of age or any concomitant medication known to prolong or shorten the QT interval.
   (c) Any clinically important abnormalities in rhythm, conduction or morphology of resting ECG e.g., complete left bundle branch block, second or third degree atrioventricular (AV) block and clinically significant sinus node dysfunction not treated with pacemaker.
13. Other cardiovascular diseases as defined by any of the following:
   (a) Symptomatic heart failure (as defined by New York Heart Association [NYHA] class ≥2),
   (b) uncontrolled hypertension,
   (c) hypertensive heart disease with significant left ventricular hypertrophy
   (d) acute coronary syndrome (ACS)/acute myocardial infarction (AMI), unstable angina pectoris, coronary intervention procedure with percutaneous coronary intervention (PCI) or coronary artery bypass grafting (CABG) within 6 months.
   (e) cardiomyopathy of any etiology
   (f) presence of clinically significant valvular heart disease
   (g) history of atrial or ventricular arrhythmia requiring treatment; subjects with atrial fibrillation and optimally controlled ventricular rate are permitted
   (h) transient ischaemic attack, or stroke within 6 months prior to screening
   (i) patients with symptomatic hypotension at screening
14. Patients with myelodysplastic syndrome (MDS)/acute myeloid leukaemia (AML) or with features suggestive of MDS/AML.
15. Refractory nausea and vomiting, chronic gastrointestinal diseases, inability to swallow the formulated product or previous significant bowel resection that would preclude adequate absorption of AZD5305
16. Known allergy or hypersensitivity to investigational product(s) or any of the excipients of the investigational product(s)
17. Any concurrent anti-cancer therapy
18. Judgment by the Investigator that the patient should not participate in the study if the patient is unlikely to comply with study procedures, restrictions and requirements
19. Any condition that, in the opinion of the Investigator, would interfere with evaluation of the study treatment or interpretation of subject safety or study results
20. Concurrent enrolment in another clinical study, unless it is an observational (non-interventional) clinical study or during the follow-up period of an interventional study
21. Involvement in the planning and/or conduct of the study (applies to both AstraZeneca staff and/or staff at the study site)
22. Previous study treatment assignment in the present study (i.e., dosing with AZD5305 previously initiated in this study)
23. Uncontrolled intercurrent illness within the last 12 months, including but not limited to, active interstitial lung disease, serious chronic GI conditions associated with diarrhoea, or psychiatric illness/social situations that would limit compliance with study requirement, substantially increase risk of incurring AEs or compromise the ability of the patient to give written informed consent.

In addition, the following are considered criteria for exclusion from the exploratory genetic research:
Previous allogeneic bone marrow transplant
Non-leukocyte depleted whole blood transfusion within 120 days of the date of the genetic sample collection

### Specific Inclusion Criteria

For inclusion in the different Parts, they must also meet the following inclusion criteria. If the criteria in the different Parts is different from that in the core criteria, the specific criteria should be followed.

In Part A (monotherapy dose-escalation):
1. Patients must have one of the following:
   (a) Histologically or cytologically confirmed relapsed advanced ovarian*, fallopian tube or primary peritoneal cancer and evidence of a predicted loss of function germline or tumour mutation in one of the following homologous recombination repair genes: BRCA1, BRCA2, PALB2, RAD51C or RAD51D
NOTE: *Patients with stromal, sex cord or germ cell tumours are excluded. Patients with carcinosarcoma are eligible.
   (b) Histologically or cytologically confirmed HER2-negative carcinoma of the breast with recurrent locally advanced or metastatic disease and evidence of a predicted loss of function germline or tumour mutation in one of the following homologous recombination repair genes: BRCA1, BRCA2, PALB2, RAD51C, or RAD51D
   (c) Histologically or cytologically confirmed advanced/metastatic CRPC and evidence of a predicted loss of function germline or tumour mutation in one of the following homologous recombination repair genes: BRCA1, BRCA2, PALB2, RAD51C, or RAD51D
   (d) Histologically or cytologically confirmed advanced/metastatic pancreatic cancer and evidence of a predicted loss of function germline or tumour mutation in one of the following homologous recombination repair genes: BRCA1, BRCA2, PALB2, RAD51C, or RAD51D
2. Patients must have evaluable disease:
   (a) At least one measurable and/or non-measurable lesions per RECIST v1.1 that can be accurately assessed at baseline and suitable for repeated assessments by CT or MRI scans; and/or
   (b) Patients with ovarian cancer will be eligible if their disease is assessable by Gynecologic Cancer InterGroup (GCIG) CA125 response criteria (pre-treatment sample must be at least twice the ULN within 2 weeks prior to starting treatment); and/or
   (c) Patients with prostate cancer will be eligible if they have PSA evaluable disease as defined by PSA levels ≥1 ng/ml.
3. Patients may have received up to one prior line of therapy with a PARPi-based regimen (either as a treatment or as maintenance)
4. In the opinion of the Investigator, patients must be suitable for treatment with a PARPi.
5. Patients receiving non-Vitamin K antagonist oral anticoagulants may be enrolled with an international normalized ratio (INR) <2; patients with other clinical causes of INR increase (such as bleeding disorders, impaired hepatic synthesis) should be excluded.

Part A dose-escalation cohorts may be expanded to evaluate further the safety, PK, PD, and biological efficacy of AZD5305.
1. Patients recruited specifically for PD evaluation must have at least 1 tumour suitable for paired biopsies and be willing to consent to pre and on-treatment biopsies.

In Part B expansions:
1. Patients must not have received prior therapy with a PARPi-based regimen (either as a treatment or as maintenance).
2. Patients must have at least one lesion, not previously irradiated (or with evidence of disease progression following radiation), that can be accurately measured at baseline as ≥10 mm in the longest diameter (except lymph nodes which must have short axis ≥15 mm) with CT or MRI and which is suitable for accurate repeated assessment as per RECIST v1.1; and/or
For patients with ovarian cancer; disease that is assessable by GCIG CA125 response criteria (pre-treatment sample must be at least twice the ULN within 2 weeks prior to starting treatment); and/or
Patients with prostate cancer will be eligible if they have PSA evaluable disease as defined by PSA levels ≥1 ng/ml.
3. Patients receiving non-Vitamin K antagonist oral anticoagulants may be enrolled with an INR <2; patients with other clinical causes of INR increase (such as bleeding disorders, impaired hepatic synthesis) should be excluded.

In Part B1 expansion (BRCA1m, BRCA2m, PALB2m, RAD51Cm or RAD51Dm HER2-negative breast cancer):
1. Patients must have metastatic or recurrent locally advanced histologically or cytologically confirmed HER2-negative carcinoma of the breast and evidence of a predicted loss of function germline or tumour mutation in either BRCA1, BRCA2, PALB2, RAD51C, or RAD51D.
2. Patients who have received platinum (cisplatin or carboplatin, either as monotherapy or in combination) for advanced breast cancer are eligible to enter the study provided there has been no evidence of disease progression during the platinum chemotherapy.
3. Patients who have received prior platinum based chemotherapy as neo-adjuvant/adjuvant treatment are eligible provided at least 12 months has elapsed between the last dose of platinum-based treatment and study treatment.

In Part B2 expansion (BRCA1m, BRCA2m, PALB2m, RAD51Cm, or RAD51Dm ovarian cancer):
1. Patients must have histologically or cytologically confirmed relapsed advanced ovarian*, fallopian tube or primary peritoneal cancer and evidence of a predicted loss of function, germline or tumour mutation in either BRCA1, BRCA2, PALB2, RAD51C, or RAD51D
   NOTE: *Patients with stromal, sex cord or germ cell tumours are excluded. Patients with carcinosarcoma are eligible.
2. Patients must have received at least one prior line of platinum-containing chemotherapy for advanced disease.
3. Subjects must be partially-platinum-sensitive (defined as progression 6 to 12 months after the end of the last platinum-based chemotherapy) or platinum-sensitive (defined as progression >12 months after the end of the last platinum-based chemotherapy);

In Part B3 expansion (ovarian cancer with high genomic instability):
1. Patients must have histologically or cytologically confirmed relapsed advanced ovarian*, fallopian tube or primary peritoneal cancer and evidence of HRD as determined by either:
   A Myriad genomic instability score (GIS) score ≥42
   An FMI global loss of heterozygosity (LoH) score ≥16%
   *Patients with stromal, sex cord or germ cell tumours are excluded. Patients with carcinosarcoma are eligible.
2. Patients must not have a known predicted loss of function germline or tumour mutation in either BRCA1, BRCA2, PALB2, RAD51C, or RAD51D.
3. Patients must have received at least one prior line of platinum-containing chemotherapy for advanced disease.
4. Patients must be partially-platinum-sensitive (defined as progression 6 to 12 months after the end of the last platinum-based chemotherapy) or platinum-sensitive (defined as progression >12 months after the end of the last platinum-based chemotherapy).

In Part B4 expansion (BRCA1/2 mutated prostate cancer):
1. Patients must have histologically or cytologically confirmed metastatic castrate-resistant prostate cancer where metastatic status is defined as at least one documented metastatic lesion on either bone scan or CT/MRI scan. Subjects whose disease spread is limited to regional pelvic lymph nodes or local recurrence (e.g., bladder, rectum) are not eligible.
2. Patients must have a known predicted loss of function germline or somatic mutation in either BRCA1 or BRCA2.
3. Subjects must have progressed on prior next-generation hormonal agent (NHA) (e.g., abiraterone acetate and/or enzalutamide) for the treatment of metastatic prostate cancer and/or CRPC. Determination of progression is done per local investigator.
4. Serum testosterone levels ≤50 ng/dL (≤1.75 nmol/L) within (≤) 28 days before treatment allocation). If the patient is being treated with luteinising hormone-releasing hormone (LHRH) agonists (patients who have not undergone orchiectomy), this therapy must have been initiated at least 4 weeks prior to Cycle 0 Day 1 and must be continued throughout the study. Subjects without prior surgical castration must be currently taking and willing to continue LHRH analogue (agonist or antagonist) therapy throughout the duration of study treatment.
5. Documented prostate cancer progression as assessed by the investigator with one of the following:
   PSA progression defined by a minimum of 3 rising PSA levels with an interval of ≥1 week between each determination. The PSA value at the screening visit should be ≥1 μg/L (1 ng/ml).
   Radiographic progression of soft tissue disease by RECIST criteria with or without PSA progression.
   Radiographic progression of bone metastasis with two or more documented new bone lesions on a bone scan with or without PSA progression.
6. Patients should not have received prior treatment with DNA-damaging cytotoxic chemotherapy, except if for a non-prostate cancer indication and last dose >5 years prior to treatment allocation. For example, subjects who have received prior mitoxantrone or platinum-based chemotherapy for prostate cancer are excluded. Prior estramustine therapy and prior use of taxanes are allowed.

In Part B5 expansion (HRRm, non-BRCA1/2 mutated prostate cancer):
1. Patients must have histologically or cytologically confirmed metastatic castrate-resistant prostate cancer where metastatic status is defined as at least one documented metastatic lesion on either bone scan or CT/MRI scan. Subjects whose disease spread is limited to regional pelvic lymph nodes or local recurrence (e.g., bladder, rectum) are not eligible.
2. Patients must have a known predicted loss of function germline or somatic mutation in one of the HRRm genes (ATM, BARD1, BRIP1, CDK12, CHEK1, CHEK2, FANCL, PALB2, RAD51B, RAD51C, RAD51D and RAD54L [without BRCA1/2m]).
3. Subjects must have progressed on prior NHA (e.g., abiraterone acetate and/or enzalutamide) for the treatment of metastatic prostate cancer and/or CRPC. Determination of progression is done per local investigator.
4. Serum testosterone levels ≤50 ng/dl (≤1.75 nmol/L) within (≤) 28 days before treatment allocation). If the patient is being treated with LHRH agonists (patients who have not undergone orchiectomy), this therapy must have been initiated at least 4 weeks prior to Cycle 0 Day 1 and must be continued throughout the study. Subjects without prior surgical castration must be currently taking and willing to continue LHRH analogue (agonist or antagonist) therapy throughout the duration of study treatment.
5. Documented prostate cancer progression as assessed by the investigator with one of the following:
   PSA progression defined by a minimum of three rising PSA levels with an interval of ≥1 week between each determination. The PSA value at the screening visit should be ≥1 μg/L (1 ng/ml).
   Radiographic progression of soft tissue disease by modified RECIST v1.1 criteria with or without PSA progression.
   Radiographic progression of bone metastasis with two or more documented new bone lesions on a bone scan with or without PSA progression.
6. Patients should not have received prior treatment with DNA-damaging cytotoxic chemotherapy, except if for a non-prostate cancer indication and last dose >5 years prior to treatment allocation. For example, subjects who have received prior mitoxantrone or platinum-based chemotherapy for prostate cancer are excluded. Prior estramustine therapy and prior use of taxanes are allowed.

### Starting Dose and Schedule

Studies with AZD5305, as well as clinically approved PARP inhibitors, have shown maintaining free plasma concentrations above the DLD-1 BRCA^{-/-} cell growth inhibition assay IC₉₅ achieves maximal pre-clinical efficacy in vivo in a mouse xenograft model (MDA-MB-436).

AZD5305 was well-tolerated in pre-clinical toxicology experiments. Based on international guidance for starting dose selection for agents in cancer patients (ICH S9), which recommends that the starting dose should be set at a dose of 1/10 of the Severely Toxic Dose in 10% of the animals (STD 10) in rodent toxicity studies, 50 mg/daily is considered a safe starting dose of AZD5305.

A dose of 10 mg/daily is predicted to give a steady state free Cₘₐₓ and AUC of 0.080 μM and 0.87 μM·h respectively (equivalent to 350 ng/mL and 3800 ng·h·mL⁻¹ total).

Each cycle will be 28 days.

### Study Treatment (AZD5305)

AZD5305 will be administered as a film-coated tablet with a unit-dose strength of 0.5 to 100 mg.

### Dose-Escalation

The dose-escalation study flow chart is presented in FIG. 1 and Table 5 and shows an example dose-escalation scheme. After the first DLT has been observed (at any dose), a Bayesian logistic regression model will be used to inform subsequent dose selections. On the basis of the pre-clinical toxicology data, allowing a start dose of up to 50 mg, rapid dose-escalation from 10 mg to 40 mg may be considered, following review of early PK and safety data.

All potential dose-escalation and de-escalation doses and schedules after the starting dose may be adjusted in light of emerging safety, tolerability, and/or PK data. A minimum of 3 patients will be recruited per cohort. Part A dose-escalation cohorts may be expanded at to evaluate further the safety, tolerability, PK, PD, and biological activity of AZD5305, provided the dose has already been declared as tolerated. Patients with specific tumour types and prior treatment status (within the current eligibility criteria) may be enrolled in these Part A expansion cohorts based on emerging data. Dose-escalation will continue in parallel. Patients recruited for tumour PD analysis will be required to have paired biopsies.

Sentinel dosing will be applied with a single patient exposed for a minimum of 24 hours before further patients are enrolled at that dose level. In the absence of any significant toxicities in the first patient as confirmed by the treating physician, the patients for the remainder of the cohort may be enrolled, concurrently or sequentially. If significant toxicities are observed during the first 24 hours of observation in the first patient of the cohort, further enrolment into the cohort will be halted until the all the safety and PK data related to that patient (if relevant) and cumulative data from all trial patients has been reviewed to determine the appropriate action.

TABLE 5
Example of dose-escalation scheme

| Cohort | AZD5305 (mg/daily) |
|---|---|
| 1 | 10 |
| 2 | 20 |
| 3 | 40 |
| 4 | 80 |
| 5 | 160 |
| Cohort n (escalate to MTD or MFD) | No more than 100% increment |

MFD = maximum feasible dose:
MTD = maximum tolerated dose;

There will not be any pause awaiting the assessment of Part A PD/PK expansion cohorts. Dose-escalation and de-escalation will follow the Bayesian adaptive design scheme below:
If no DLT at any dose has been observed, then dose-escalation may occur without modelling.
After the first DLT has been observed (at any dose), a Bayesian logistic regression model will be used to inform subsequent dose selections. After each cohort, cumulative data on the evaluable patients will be used to estimate the predicted probability of a DLT at each potential next dose.
The recommendation for the next dose will be based on the following principles:
The recommendation for the next dose will be chosen to target a DLT of 25% for monotherapy but ignoring any dose for which the model suggests the probability of a DLT exceeds 33%. This will allow efficient identification of the MTD whilst restricting the risk of overdosing.

Dose-escalation and de-escalation will be completed when any of the following occur:
The target toxicity of 25% (for monotherapy) is reached and the required precision of the estimated MTD is achieved (i.e., the ratio of the upper/lower 95% credible interval limits for the estimated MTD <5.0).
The maximum number of evaluable patients for the dose-escalation cohort has been reached.
The MFD is achieved.
Decision made to stop dose-escalation.

Prior established dose toxicity relationship at doses at which 10% and 50% (or similar) of patients experience DLTs will be used to inform the Bayesian model.
1. There is no minimum time period required between completion of dosing in the last evaluable patient from 1 cohort and the start of dosing in the subsequent cohort.
2. Intermediate dose levels, dosing intervals and/or frequency, and/or schedules may be evaluated prior to declaring the RP2D, which will be guided by the evolving safety, preliminary efficacy, PK and PD data.

### Dose-Limiting Toxicity

DLTs will be evaluated during Cycle 0 and Cycle 1 of treatment. Toxicity will be graded according to the CTCAE National Cancer Institute (NCI) v5.0.

A DLT is defined as any toxicity that occurs from the first dose of study treatment up to and including the planned end of Cycle 1 (the DLT assessment period) that is assessed as unrelated to the disease or disease-related processes under investigation and which includes:

Haematological toxicities including:
Grade 4 neutropenia (ANC <500 cells/mm³) lasting >4 days; Grade 3 or above neutropenia (ANC <1000 cells/mm³) of any duration accompanied with a single temperature of >38.3° C. or a sustained temperature of ≥38.0° C. (for more than one hour) and/or systemic infection.
Grade 4 thrombocytopenia (<25,000/mm³) lasting >1 day or Grade 3 or above thrombocytopenia (<50,000/mm³) with clinically significant bleeding attributable to thrombocytopenia.
Any other confirmed haematological toxicity ≥CTCAE Grade 4 (a repeat may be required for confirmation of an isolated abnormality in the absence of clinical signs, symptoms or other abnormal investigations, i.e., a suspected spurious value).
Haematological AEs requiring dose interruption for ≥14 days

Cardiac DLTs including:
symptomatic tachycardia or tachycardia with resting supine heart rate of >125 beats per minute persisting for at least 10 minutes
hypotension requiring medical intervention e.g., IV fluids
any other cardiac toxicity of CTCAE >Grade 2
QTcF interval value <340 milliseconds confirmed on at least 2 separate ECGs, recorded 5 minutes apart
QTcF prolongation >500 milliseconds or QTcF prolongation from baseline by >60 milliseconds confirmed on at least 2 separate ECGs, recorded 5 minutes apart

Non-haematological toxicities (CTCAE Grade ≥3) including:
Nausea, vomiting or diarrhoea lasting for ≥72 hours despite administration of maximal supportive therapy.
Any treatment-related event, including significant dose reductions, omissions or delays, judged to be a DLT. Examples may include confirmed laboratory abnormalities (CTCAE Grade ≥3 or above), CTCAE Grade 2 toxicities that are clinically significant and/or unacceptable according to the Investigator, toxicities that result in an inability to administer at least 75% of study treatment during Cycle 1 or delay the administration of study treatment in the subsequent cycle by ≥7 consecutive days.
Any death not clearly due to the underlying disease or extraneous causes

DLTs will not include the following:
Transient isolated laboratory abnormalities which are not considered clinically significant and resolve to baseline within 72 hours without any intervention.
Alopecia
CTCAE Grade ≥3 nausea, vomiting or diarrhoea lasting <72 hours in the absence of maximum supportive therapy
Grade 3 fatigue <5 days.
Grade 3 hypertension in the absence of maximal medical therapy.
Grade 3 electrolyte abnormalities that resolve to Grade ≤1 within 48 hours spontaneously or with conventional medical intervention and that are not clinically complicated.
Grade 3 rash that resolves to Grade ≤1 within 3 weeks.
Grade 3 or Grade 4 elevation in serum amylase and/or lipase that are not associated with clinical or radiographic evidence of pancreatitis

In order to define a DLT, patients should not be prophylactically prescribed growth factor support, blood products, antiemetics, anti-diarrhoeals or antipyretics during Cycle 1 of therapy, unless this is part of the standard supportive medications required for combination agents.

### Definition of DLT-Evaluable Patients

For decisions on dose-escalation (Part A), an evaluable patient is defined as a patient that has received AZD5305 and either:
Has completed the safety follow-up and has received at least 75% of the total amount of the planned dose of AZD5305 through the DLT assessment period; or
Has experienced a DLT during the DLT assessment period.

### Definition of MTD

The MTD is defined as the highest dose at which the predicted probability of a DLT is less than or equal to 25% (for monotherapy) or 30% (for combinations). At least 6 evaluable patients are required to determine the MTD.

### Definition of Maximum Feasible Dose (MFD)

A dose and schedule will be considered non-feasible and escalation will cease where PK parameters determine that a maximum absorbable dose has been reached, or where the maximum dose permitted based on the Chemistry Manufacturing and Controls (CMC) quality specifications (currently set against a maximum daily dose of 320 mg) has been reached, whichever is the lower dose.

### Definition of Predicted Effective Dose

Determination of the predicted effective dose (PED) will be based on pre-clinical PK/PD modelling and human PK. It is defined as the AZD5305 dose required to achieve a minimum steady-state plasma drug concentration (C_{min,ss}) above 28 ng/ml (total plasma, IC₉₅ DLD-1 BRCA2−/−) in the majority of patients. If C_{min,ss} is not available, other PK parameters including area under the curve (AUC) and maximum plasma drug concentration (Cₘₐₓ) could be used based on emerging clinical data; additionally, alternate dosing schedules that achieve similar target engagement may also be evaluated.

### Definition of Recommended Phase 2 Dose (RP2D)

The RP2D will take into account the MTD (or alternative doses/schedules in the absence of MTD), MFD, PED, PK, and biological/clinical activity (PD data), as well as data beyond Cycle 1 during dose-escalations. The RP2D will not exceed the maximum tolerated dose.

Collection of Plasma Samples for ctDNA

A peripheral blood sample will be collected to provide plasma for ctDNA.

Testing may include (but is not limited to):
Whether there is sufficient ctDNA in plasma for mutational testing
Correlation of baseline ctDNA levels and kinetics of ctDNA changes with clinical outcome measures
Correlation between tumour and plasma mutational status
Genetic alterations as potential mechanisms of sensitivity or resistance to AZD5305 treatment
Future diagnostic development

### Efficacy Assessments

### RECIST v1.1 Assessments of Tumour

The RECIST v1.1 guidelines for measurable and non-measurable TLs and non-target lesions (NTLs) and the objective tumour response criteria can be found in Eisenhauer 2009.

At baseline, the imaging modalities used for assessment should be contrast enhanced computer tomography (CT) (magnetic resonance imaging [MRI] where CT is contraindicated) scans of the chest, abdomen and pelvis (including liver and adrenal glands) and should encompass all areas of known predilection for metastases in the disease under evaluation, and should additionally investigate areas that may be involved based on signs and symptoms of individual patients. Brain imaging (either with CT or MRI) is only required at baseline in patients with known or suspected brain metastases. Follow-up CT or MRI assessments will cover chest, abdomen and pelvis with any other regions imaged at baseline where disease was present. Any other sites at which new disease is suspected should also be appropriately imaged. Radiological examinations performed in the conduct of this study should be retained at site as source data. All treatment decisions will be based on site assessment of scans.

Baseline radiological assessments should be performed no more than 28 days before the start of study treatment, and ideally should be performed as close as possible to the start of study treatment. Scans obtained as part of standard clinical practice, prior to informed consent, but within the 28-day period are acceptable. The radiological confirmatory scans should be performed no less than 4 weeks after the prior assessment of tumour and preferably at the next scheduled visit (in the absence of clinically significant deterioration). The methods of assessment used at baseline should be used at each subsequent follow-up assessment through to objective confirmed radiological disease progression, as defined by Investigator assessed RECIST v1.1 or PCWG3 for patients with prostate cancer. Tumour assessments should be performed every 8 weeks from Cycle 1 Day 1 (±1 week) or earlier, if disease progression is suspected. If a patient discontinues treatment (and/or receives a subsequent cancer therapy) prior to progression then the patient should continue to be followed (unless they withdraw consent) until objective disease progression, as defined by RECIST v1.1 or PCWG3 for patients with prostate cancer. Scans confirming progression should not be conducted within 1 week after a progression biopsy to allow for reduction in inflammation.

If scans are performed outside of scheduled visit window interval and the patient has not progressed, every attempt should be made to perform the subsequent scans at their scheduled visits whilst the patient remains on study treatment. If the patient interrupts treatment or incurs a treatment delay, scans should continue to occur at the protocol-defined frequency.

It is important to follow the schedule of assessments as closely as possible.

### Bone Scans

Patients with breast cancer should have a baseline bone scan performed in the screening period (28 days) prior to first dose of study treatment (AZD5305) and as close as possible to study treatment start. At follow-up, additional bone scans may be performed, if clinically indicated and should be performed at disease progression.

All prostate cancer patients should have a baseline bone scan in the screening period (28 days) prior to first dose. Follow-up bone scans should then be performed 8-weekly until disease progression for all prostate cancer patients.

### Tumour Evaluation by RECIST v1.1

Categorisation of objective tumour response assessment will be based on the RECIST v1.1 criteria of response: CR, PR, and stable disease (SD). The CR and PR will need to be confirmed. Target lesion (TL) progression will be calculated in comparison to when the tumour burden was at a minimum (i.e., smallest sum of diameters previously recorded on study). In the absence of progression, tumour response will be calculated in comparison to the pre-dose tumour measurements obtained before starting treatment.

If the Investigator is in doubt as to whether progression has occurred, particularly with response to NTLs or the appearance of a new lesion, it is advisable to continue treatment until the Investigator reassesses the patient's status at the next scheduled assessment (confirmation) or sooner if clinically indicated.

To achieve ‘unequivocal progression’ based on non-target disease, there must be an overall level of substantial worsening in non-target disease such that, even in presence of SD or PR in target disease, the overall tumour burden has increased sufficiently to merit discontinuation of therapy. A modest increase in the size of 1 or more NTLs is usually not sufficient to qualify for unequivocal disease progression status.

### Bone Lesion Assessment by PCWG3 (Applicable to Prostate Cancer Patients Only)

In patients with prostate cancer, disease progression will be deemed to have occurred if one or more of the following criteria is met:
Soft tissue disease progression as defined by RECIST v1.1 (Table 6)
Bone lesion progression by Prostate Cancer Working Group 3 (PCWG3) (Table 6)
Death

In patients with prostate cancer, categorisation of tumour progression of bone lesions will be based on the PCWG3 criteria. Positive hot spots on the bone scan should be considered significant and unequivocal sites of malignant disease to be recorded as metastatic bone lesions. When the bone scan is the sole indicator of progression, radiographic progression based on the appearance of new bone lesions is defined as:

At least two new lesions observed at the first post-baseline bone scan, with at least two additional new lesions observed at a subsequent bone scan (2+2 rule), or Following the first post-baseline bone scan, at least 2 new lesions relative to the first post-baseline scan confirmed on a subsequent scan at least 6 weeks apart, as specified in PCWG3 criteria (Scher 2016). In situations where the scan findings are suggestive of a flare reaction, or apparent new lesion(s) may represent trauma, confirmation with other imaging modalities such as MRI (magnetic resonance imaging) or fine-cut CT (computer tomography) is required. The exception to this requirement occurs if multiple new areas of uptake are observed.

The requirements for determination and confirmation of radiographic progression by either bone scan (bone progression) or CT/MRI (soft tissue progression) are summarised in Table 6:

TABLE 6
Requirements for Documentation of Disease Progression in
Patients with Prostate Cancer

| Visit | Criteria for bone progression | | Criteria for soft tissue | |
|---|---|---|---|---|
| date | (PCWG3) | | progression (RECIST v1.1) | |
| Week | 3. | 2 or more new lesions | 5. | Progressive disease on |
| 8 | | compared to baseline | | CT or MRI by RECIST |
| | | bone scan | | v1.1 |
| | 4. | Requires confirmation | 6. | No confirmation scan |
| | | scan at least 6 weeks later | | required |
| | | with ≥2 additional lesions | | |
| | | compared to Week 8 scan | | |
| Week | 7. | 2 or more new lesions | 9. | Progressive disease on |
| 16 or | | compared to Week 8 | | CT or MRI by RECIST |
| later | | bone scan | | v1.1 |
| | | 8. Requires confirmation | 10. | No confirmation scan |
| | | scan at least 6 weeks later | | required |
| | | for persistence or increase | | |
| | | in number of lesions | | |

### Safety

All safety analyses will be performed on the safety analysis set. At the end of the study, appropriate summaries of all safety data will be produced, as defined below.

Data from all cycles of initial treatment will be combined in the presentation of safety data. Graphical presentations of safety data may be presented as is deemed appropriate. This may include, but is not restricted to, presentation of parameters against time, concentration or shift plots. Appropriate scatter plots may also be considered to investigate trends in parameters compared to baseline.

### Exposure

Exposure to each study treatment, i.e., total amount of study treatment received will be listed for all patients. Total treatment duration and actual treatment duration (=total treatment duration excluding dose interruptions not in accordance with the protocol) will be summarised for each study treatment and cohort by the following: mean, standard deviation, minimum, maximum, median and number of observations. In addition, the number and percentage of patients with at least 1 non-protocolled dose interruption and at least 1 dose reduction will be presented.

### Adverse Events

AEs will be listed individually by patient and cohort. For patients who have a dose modification, all AEs (due to study treatment or otherwise) will be assigned to the initial dose group.

The number of patients experiencing each AE will be summarised by the Medical Dictionary for Regulatory Activities (MedDRA) system organ class, MedDRA preferred term and CTCAE Grade. The number and percentage of patients with AEs in different categories (e.g., causally related, CTCAE Grade ≥3 etc.) will be summarised by dose regimen, and events in each category will be further summarised by MedDRA system organ class and preferred term, by dose group. SAEs will be summarised separately.

Any AE occurring before the first dose of study treatment (i.e., before study Day 1) will be included in the data listings but will not be included in the summary tables of AEs. Treatment-emergent AEs occurring prior to first dose of study treatment (i.e., before study Day 1) which subsequently worsen in severity following dosing will be included in the summary tables.

Any AE occurring within the defined 28-day follow-up period after discontinuation of study treatment will be included in the AE summaries. Any AEs in this period that occur after a patient has received further therapy for cancer (following discontinuation of study treatment) will be flagged in the data listings. AEs occurring after the 28-day follow-up period after discontinuation of study treatment will be listed separately, but not included in the summaries.

### Other Safety Data

Hematology, clinical chemistry, vital signs, ECG data, ECOG PS, physical examination, demographic data, medical histories and concomitant medications will be listed individually by patient and suitably summarised. For all laboratory variables, which are included in the current version of CTCAE, the CTCAE Grade will be calculated. Summary statistics of mean, median, standard deviation, minimum, maximum and number of observations will be used for continuous variables. Categorical variables will be summarised by frequency counts and percentages for each category (n [%]).

Details of any deaths will be listed for all patients.

### Efficacy

### Tumour Response Data

Tumour response data will be summarised for dosed patients with measurable disease at baseline, except for PFS that will be summarised for dosed patients.

Data will be listed and summarised by cohort using the following response categories: CR, PR, SD, disease progression and not evaluable (NE).

Waterfall plots (bar charts) indicating the percentage best change from baseline in sum of the diameters of TLs may be produced by cohort.

Tumour response includes the following variables:
Percentage best change in TL
DoR
PFS
ORR
TTR

Additional tumour response criteria based on tumour markers may be considered for sensitivity analyses.

### Derivation of Tumour Response Variables

Every 8 weeks (±1 week), or earlier if disease progression is suspected, patients will be programmatically assigned a RECIST v1.1 visit response of CR, PR, SD, and disease progression depending on the status of their disease compared to baseline and previous assessments.

Progression of TLs will be calculated in comparison to when the tumour burden was at a minimum (i.e., smallest sum of diameters previously recorded during the study). In the absence of progression, tumour response (CR, PR, SD) will be calculated in comparison to the baseline tumour measurements obtained before starting treatment.

If a patient has had a tumour assessment, which cannot be evaluated, then the patient will be assigned a response of NE unless there is evidence of progression in which case the response will be assigned as disease progression.

### Percentage Best Change in TL

Percentage change in tumour size will be determined for patients with measurable disease at baseline and is derived at each visit by the percentage change in the sum of the diameters of TLs.

### Duration of Response (DoR)

Duration of response is defined as the time from the date of first documented response (which is subsequently confirmed) until date of documented progression or death in the absence of disease progression (PD) (i.e., date of PFS event or censoring-date of first response+1). The end of response should coincide with the date of progression or death from any cause used for the PFS endpoint. If a patient does not progress following a response, then their duration of response will use the PFS censoring time.

### Time to Response (TTR)

The time of the initial response will be defined as the latest of the dates contributing towards the first visit that was PR or CR that was subsequently confirmed.

### Progression-Free Survival (PFS)

PFS is defined as the time from Cycle 1 Day 1 until the date of objective disease progression or death (by any cause in the absence of progression) regardless of whether the patient withdraws from study therapy or receives another anti-cancer therapy prior to progression (i.e., date of PFS event or censoring—date of first dose+1). Patients who have not progressed or died at the time of analysis will be censored at the time of the latest date of assessment from their last evaluable RECIST v1.1 assessment. However, if the patient progresses or dies after 2 or more missed visits, the patient will be censored at the time of the latest evaluable RECIST v1.1 assessment prior to the 2 missed visits.

### Objective Response Rate (ORR)

ORR will be assessed per RECIST v1.1. ORR is defined as the percentage of patients who have a confirmed visit response of CR or PR prior to any evidence of progression (as defined by RECIST v1.1).

For the analysis of ORR, the response evaluable set will be derived and will exclude patients who do not have measurable disease at baseline. Note that responses that occur after the start of subsequent anti-cancer therapy must be excluded from the derivation of ORR.

A visit response of CR is defined when all TLs and NTLs present at baseline have disappeared (with the exception of lymph nodes which must be <10 mm to be considered non-pathological) and no new lesions have developed since baseline. A visit response of PR is defined when the sum of diameters of the TLs has decreased by 30% or more compared to baseline (with no evidence of progression) and the NTLs are at least stable with no evidence of new lesions.

In the case of SD, measurements should have met the SD criteria at least once after the study start.

When the Investigator reassesses the progression of the patient at a later date, the date of the initial scan should be declared as the date of progression if the repeat scans confirm progression.

### CA125 Response (Ovarian Cancer Patients)

The endpoint CA125 response is defined as at least a 50% reduction in CA125 levels from a pre-treatment sample. The response must be confirmed and maintained for at least 28 days. Patients can be evaluated according to CA125 only if they have a pre-treatment sample that is at least twice the upper limit of normal and within 2 weeks prior to starting treatment.

### Pharmacokinetics

Following single dose and following multiple dose administration, plasma concentrations of AZD5305 and the following PK parameters may be determined (including but not limited to): AUC, Cₘₐₓ, and Tₘₐₓ as data allow.

PK analysis of plasma concentration data for AZD5305 will be performed using actual elapsed sampling times and standard non compartmental methods.

Venous blood samples (2.0 mL each) for determination of concentrations of AZD5305 in plasma will be taken at the times presented in Table 7. Time points post-dose are relative to the timing of AZD5305 dose.

Urine samples (10 mL) for the determination of concentrations of AZD5305 will be taken from the total urine sample provided during each of the collection intervals presented in Table 7. The date and time of collection and the date and time of freezing of each sample will be recorded. Additionally, the total volume/weight of each urine collection will be recorded.

TABLE 7
Blood and urine sampling schedule for AZD5305

| | Time for | Collection of intervals |
|---|---|---|
| Cycle and Day | Blood samples | for urine samples |
| Cycle 0 Day 1 & Cycle 1 | Pre dose | Pre-dose (spot urine) |
| Day 15 | | |
| Cycle 0 Day 1 & Cycle 1 | 30 min | 0 to 4 h |
| Day 15 | post-dose | |
| Cycle 0 Day 1 & Cycle 1 | 1 h post-dose | |
| Day 15 | | |
| Cycle 0 Day 1 & Cycle 1 | 1.5 h post-dose | |
| Day 15 | | |
| Cycle 0 Day 1 & Cycle 1 | 2 h post-dose | |
| Day 15 | | |
| Cycle 0 Day 1 & Cycle 1 | 3 h post-dose | |
| Day 15 | | |
| Cycle 0 Day 1 & Cycle 1 | 4 h post-dose | 4 to 8 h |
| Day 15 | | |
| Cycle 0 Day 1 & Cycle 1 | 6 h post-dose | |
| Day 15 | | |
| Cycle 0 Day 1 & Cycle 1 | 8 h post-dose | 8 to 24 h ª |
| Day 15 | | |
| Cycle 0 Day 2 & Cycle 1 | 24 h post-dose | |
| Day 16 | | |
| Cycle 0 Day 3 | 48 h post-dose | 24 to 48 h ª |
| Cycle 1 Day 1, Cycle 1 | Pre dose and | None |
| Day 8, Cycle 2 Day 1 and | 1 to 4 h | |
| Cycle 3 Day 1 | post-dose | |
| Cycle 1 Day 8-15 | 24 h post-dose | None |
| | (to match biopsy | |
| | collection) b | |
| Discontinuation/EoT | Discontinuation/ | None |
| | EoT visit | |

EoT = end of treatment;
h = hour(s)
^{a} Urine collected at home
^{b} Sample only collected if biopsy is performed

### Pharmacodynamics

The change from baseline in PD biomarkers, for example poly-ADP-ribosylation (PAR) will be assessed, as well as other PD parameters, as data allow, and will be described on the PD analysis set.

The collection of blood-based PD biomarker samples is mandatory to obtain a preliminary assessment of PARP1 target inhibition in patients. Peripheral blood samples will be collected as detailed in Table 8. In addition, there will be PK samples collected at the same time as PD samples to allow exploration of the exposure/PD relationships. The exact time of PD sample collection will be noted. The collection of the PD samples may be adjusted during the study and according to local regulatory approval, dependent on emerging data, in order to ensure appropriate characteristics of the plasma concentration-time profile.

TABLE 8
Pharmacodynamic blood sample collection

| | | Time for | |
|---|---|---|---|
| | | Blood | |
| | | samples for | |
| | Time for Blood | PD (Immune | Time for |
| | samples for PD | markers, | Blood samples |
| Cycle and Day | (PAR/PARP) | proteomics) | for ctDNA |
| Screening | Post-consent | None | Post-consent |
| Cycle 0 Day 1 & | Pre-dose | Pre-dose | None |
| Cycle 1 Day 15 | | | |
| Cycle 0 Day 1 & | 1 h post-dose | None | None |
| Cycle 1 Day 15 | | | |
| Cycle 0 Day 1 & | 4 h post-dose | None | None |
| Cycle 1 Day 15 | | | |
| Cycle 0 Day 2, | 24 h post-dose | None | None |
| Cycle 1 Day 16 | | | |
| Cycle 0 Day 3 | 48 h post-dose | None | None |
| Cycle 1 Day 1, | Pre-dose & 1-4 h | Pre-dose at | Pre-dose at |
| Cycle 1 Day 8, | post-dose | Cycle 1 Day 1 | Cycle 1 |
| and | | and Cycle 1 | Day 1, Cycle 1 |
| Cycle 1 Day 15 | | Day 15 | Day 8 and |
| | | | Cycle 1 Day 15 |
| Cycle 2 Day 1 | Pre-dose & 1-4 h | Pre-dose | Pre-dose |
| | post-dose | | |
| Cycle 2 Day 15 | None | None | Pre-dose |
| Cycle 3 Day 1 | Pre-dose & 1-4 h | Pre dose | Pre-dose |
| | post-dose | | |
| Cycle 4 onwards | None | Pre-dose | At each |
| | | Day 1, | radiological |
| | | Pre-dose up to | assessment |
| | | Cycle 5 Day 1 | |
| Cycle 1 Day 8-15 | 24 h post-dose | None | None |
| | (to match PK | | |
| | collection) ^{a} | | |
| Discontinuation | At | None | At |
| (EoT) | discontinuation/ | | discontinuation/ |
| | EoT visit | | EoT visit |

ctDNA = circulating tumour deoxyribonucleic acid;
EoT = end of treatment;
h = hour(s);
PAR = poly adenosine diphosphate-ribose;
PARP = poly adenosine diphosphate-ribose (ADP-ribose) polymerase;
PD = pharmacodynamic;
PK = pharmacokinetic
^{c} Sample only collected if biopsy is performed

### Exploratory Analyses

Where appropriate, tumour markers will be collected e.g., CA125, prostate specific antigen (PSA), CA19.9 and CA15.3 from patients with ovarian, prostate, pancreatic and breast cancer, respectively.

The efficacy analysis (ORR, TTR, PFS, DoR) will be repeated for various biomarker subgroups defined at baseline: tBRCA, gBRCA, sBRCA, BRCA methylation, non-BRCAm, HRR (individually and as a panel), genomic instability status, RAD51 nuclear foci. Exploratory analyses will be described in the SAP or in dedicated analysis plans according to the exploratory endpoints. The results of exploratory analyses may be reported separately from the CSR.

### Collection of PBMCs

PBMCs will be assessed for PD changes in biomarkers that may include but are not limited to protein/gene levels and post-translational modifications such as poly (ADP-ribose). Peripheral whole blood subpopulations may also be enumerated to determine any PD effects of potential immunological relevance, such as changes in the number of B-lymphocytes or regulatory T-lymphocytes or evaluated for T-cell receptor clonality or gene expression changes.

The number of samples taken, as well as the volume required for each analysis, may be changed during the study as new data on AZD5305 becomes available.

Collection of Plasma Samples for ctDNA and other Soluble Analytes

A peripheral blood sample will be collected to provide plasma for ctDNA to assess clinical response and to interrogate changes in genetic alterations as potential mechanisms of sensitivity/resistance to AZD5305 treatment. Blood samples for ctDNA are to be collected as detailed in Table 8. Other soluble analytes in plasma such as proteins, cytokines may be analysed for downstream effects of PARP1 inhibition.

### Collection of Tumour Samples for PD Assessment

Paired fresh tumour biopsies for PD assessment are optional for all patients in Module 1 except for patients recruited to a Part A cohort expansion for PD assessment, in which case they are mandatory. Paired biopsies will be collected within 15 days prior to Cycle 0 Day 1 and during the on-treatment period (any time in Cycle 1 between Days 8 and 15 at 24 hours post dose).

Accessible lesions are defined as tumour lesions which are amenable to repeat biopsy, unless clinically contraindicated or the patient has withdrawn consent. Failure to obtain sufficient tumour sample after making best efforts to biopsy the tumour will not be considered a protocol deviation.

Blood samples for PK will also be taken at the time of any on-treatment biopsy sample. Biopsy samples will be analysed for the effects of PARP1 inhibition in tumour and may include but is not limited to the assessment of the following biomarkers: poly (ADP-ribose), PARP-DNA trapping, pH2AX (Ser139) and RAD51 foci. On-treatment biopsy timing may be refined with emerging PK and/or PD data during the course of the study.

### Results

The following results are derived from the above studies carried out on 61 patients dosed from 10 mg to 140 mg QD. The data cut-off date is 22 Feb. 2022.

The patients can be characterised as follows:

| Characteristics | Total (N = 61) | |
|---|---|---|
| Median age (range), yrs | 56.0 | (25, 78) |
| Age ≥65 years, n (%) | 16 | (26.2) |
| Male/female, n (%) | 20 | (32.8)/ |
| | 41 | (67.2) |
| Race, n (%) | | |
| White | 40 | (65.6) |
| Asian | 11 | (18.0) |
| Black/African American | 3 | (4.9) |
| Other | 4 | (6.6) |
| Pending | 3 | (4.9) |
| ECOG PS 0/1, n (%) | 25 | (41.0)/ |
| | 36 | (59.0) |
| Tumor type, n (%) | | |
| Ovarian | 19 | (31.1) |
| Platinum sensitive (PD ≥6 mos post-platinum) | 4 | (21.1) |
| Platinum Resistant/Refractory | 14 | (73.7) |
| Pending | 1 | (5.3) |
| Breast | 18 | (29.5) |
| TNBC (triple-negative breast cancer) | 5 | (27.8) |
| HR+ | 12 | (66.7) |
| Pending | 1 | (5.6) |
| Pancreatic | 12 | (19.7) |
| Prostate | 10 | (16.4) |
| Pending¹ | 2 | (3.3) |
| Alteration for eligibility (by eCRF), n (%) | | |
| BRCA1 | 21 | (34.4) |
| BRCA2 | 29 | (47.5) |
| PALB2 | 7 | (11.5) |
| RAD51C | 1 | (1.6) |
| RAD51D | 0 | |
| Pending | 3 | (4.9) |
| Median number of prior lines of therapy (range) | 3 | (0, 8) |
| Prior PARPi/platinum therapy status, n (%) | | |
| PARPi and/or platinum | 44 | (72.1) |
| Both PARPi and platinum | 18 | (40.9) |
| Platinum only | 20 | (45.5) |
| PARPi only | 6 | (13.6) |
| No PARPi or platinum | 17 | (27.9) |

eCRF, electronic case report form; HR, hormone receptor

| Dose | Number of Patients |
|---|---|
| 10 mg | 8 |
| 20 mg | 19 |
| 40 mg | 17 |
| 60 mg | 10 |
| 90 mg | 3 |
| 140 mg | 4 |

### Plasma Concentration

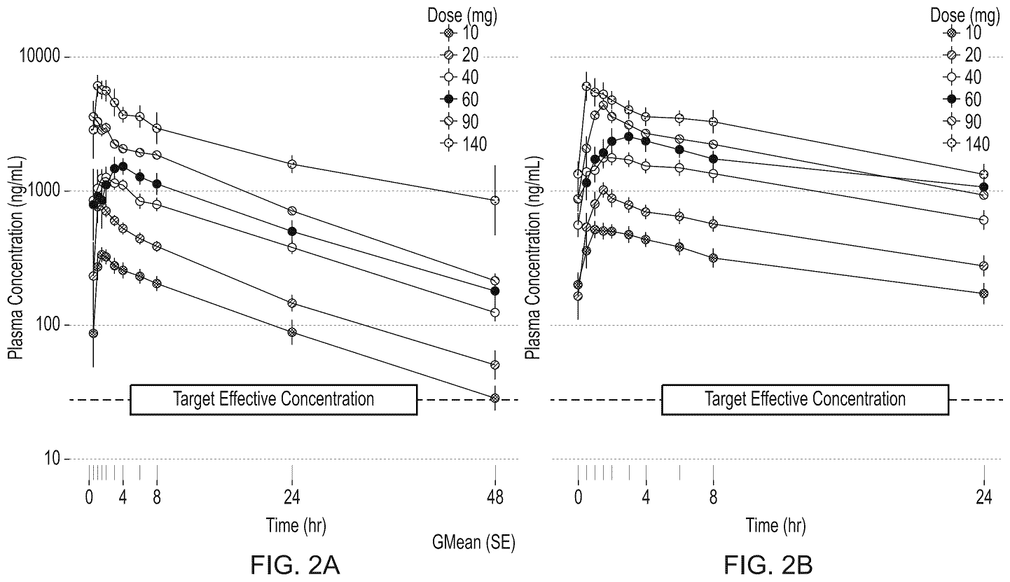

FIG. 2A shows the AZD5305 plasma concentration against the time after a single dose. FIG. 2B shows the AZD5305 plasma concentration against the time after a dose during continuous dosing (cycle 1 Day 15).

| Dose | Symbol | Dose | Symbol |
|---|---|---|---|
| 10 mg | [symbol] | 60 mg | [symbol] |
| 20 mg | [symbol] | 90 mg | [symbol] |
| 40 mg | [symbol] | 140 mg | [symbol] |

Dose proportional increase in exposure (Cₘₐₓ and AUC) was observed with increasing dose (10-140 mg QD). There was quick onset (Tₘₐₓ 0.5-3 h) with mean terminal elimination half-life 13.1-16.4 hours across the different cohorts. Steady state Cₘᵢₙ above target effective concentration (TEC) was achieved in all patients with mean fold Cₘᵢₙ/TEC 7.12 and 55.88 at 10 mg and 140 mg QD, respectively

TEC, target effective concentration; TEC: IC₉₅ in DLD-1 BRAC2−/− Safety summary

| | AZD5305 | | AZD5305 | | AZD5305 | | AZD5305 | |
|---|---|---|---|---|---|---|---|---|
| | 10 mg QD | | 20 mg QD | | 40 mg QD | | 60 mg QD | |
| AE category, n (%) | (n = 8) | | (n = 19) | | (n = 17) | | (n = 10) | |
| Duration of therapy | 4.8 | (1.8-14.9) | 2.8 | (0.9-11.5) | 2.0 | (0.4-9.6) | 1.95 | (0-3.8) |
| (months), median (range) | | | | | | | | |
| All TEAEs | 8 | (100.0) | 19 | (100.0) | 17 | (100.0) | 8 | (80.0) |
| Grade ≥3 AEs | 3 | (37.5) | 10 | (52.6) | 6 | (35.3) | 3 | (30.0) |
| Serious TEAEs | 0 | | 9 | (47.4) | 5 | (29.4) | 2 | (20.0) |
| Discontinuations | 0 | | 0 | | 0 | | 1 | (10.0) |
| Dose reductions | 1 | (12.5) | 0 | | 1 | (5.9) | 0 | |
| AZD5305-related TEAEs | 5 | (62.5) | 11 | (57.9) | 9 | (52.9) | 4 | (40.0) |

| | | | | | | | |
|---|---|---|---|---|---|---|---|
| Grade ≥3 AEs | 3 | (37.5) | 3 | (15.8) | 1 | (5.9) | 0 |

| Serious TEAEs | 0 | | 0 | 0 | | 0 |
|---|---|---|---|---|---|---|
| Discontinuations | 0 | | 0 | 0 | | 0 |
| Dose reductions | 1 | (12.5) | 0 | 1 | (5.9) | 0 |

| | AZD5305 | | AZD5305 | | | |
|---|---|---|---|---|---|---|
| | 90 mg QD | | 140 mg QD | | Total | |
| AE category, n (%) | (n = 3) | | (n = 4) | | (N = 61) | |
| Duration of therapy | 4.8 | (4.6-4.9) | 0.7 | (0.6-1.6) | 2.1 | (0-14.9) |

| (months), median (range) | | | | | |
|---|---|---|---|---|---|
| All TEAEs | 3 | (100.0) | 0 | 55 | (90.2) |
| Grade ≥3 AEs | 2 | (66.7) | 0 | 24 | (39.3) |
| Serious TEAEs | 0 | | 0 | 16 | (26.2) |
| Discontinuations | 0 | | 0 | 1 | (1.6) |
| Dose reductions | 0 | | 0 | 2 | (3.3) |
| AZD5305-related TEAEs | 3 | (100.0) | 0 | 32 | (52.5) |
| Grade ≥3 AEs | 2 | (66.7) | 0 | 9 | (14.8) |

| Serious TEAEs | 0 | 0 | 0 | |
|---|---|---|---|---|
| Discontinuations | 0 | 0 | 0 | |
| Dose reductions | 0 | 0 | 2 | (3.3) |

AZD5305 was well tolerated across all doses, with only 2 patients (3.3%) having dose reductions (for grade 3 neutropenia and grade 1 thrombocytopenia related to AZD5305). At the data cut-off, there had been no DLTs and no AZD5305-related serious AEs or treatment discontinuations.

### PARP1 Inhibition

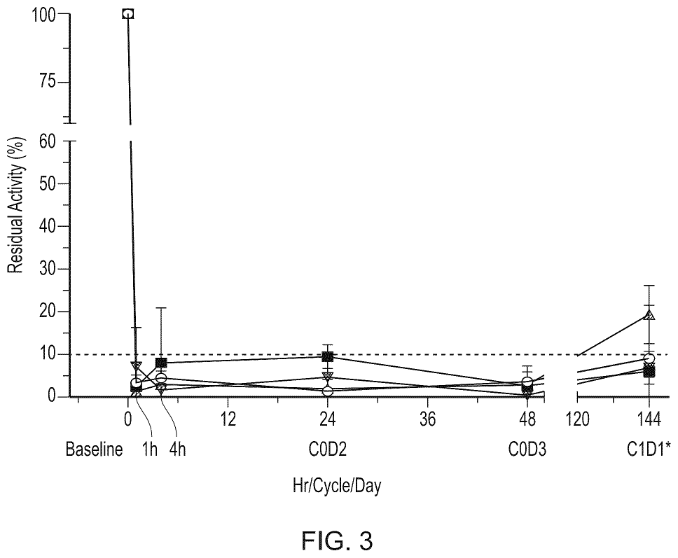

Pharmacodynamic analyses were carried out on PBMCs to measure PARP1 inhibition by the loss of signal from poly (ADP-ribose) after a single dose of AZD5305. PARylation inhibition has been a useful measure of target engagement for first generation PARP inhibitors. The results are shown in FIG. 3, in which:

| Dose | n | Symbol |
|---|---|---|
| 10 mg | 2 | ● |
| 20 mg | 3 | ▪ |
| 40 mg | 2 | ▴ |
| 60 mg | 2 | ▾ |

C1D1 was median 1 day after C0D3 (max 4 days post C0D3)

Durable PARylation inhibition ≥90% was observed at all tested doses (10, 20, 40, and 60 mg) in PBMCs of 9 patients after a single dose of AZD5305

### Efficacy Assessments

46 patients were included in the interim response analysis set, which represents all dosed subjects who had measurable disease at baseline and who received their first dose at least 13 weeks prior to data extract. 6 patients were not evaluable for RECIST v1.1 assessment—5 did not have a follow up scan and 1 had SD <7 weeks. Of 40 evaluable patients, by RECIST v1.1, observed were:
10 Partial Responses (PR) (7 confirmed; 3 unconfirmed)
11 Stable Disease (SD), and
19 Progressive Disease (PD)

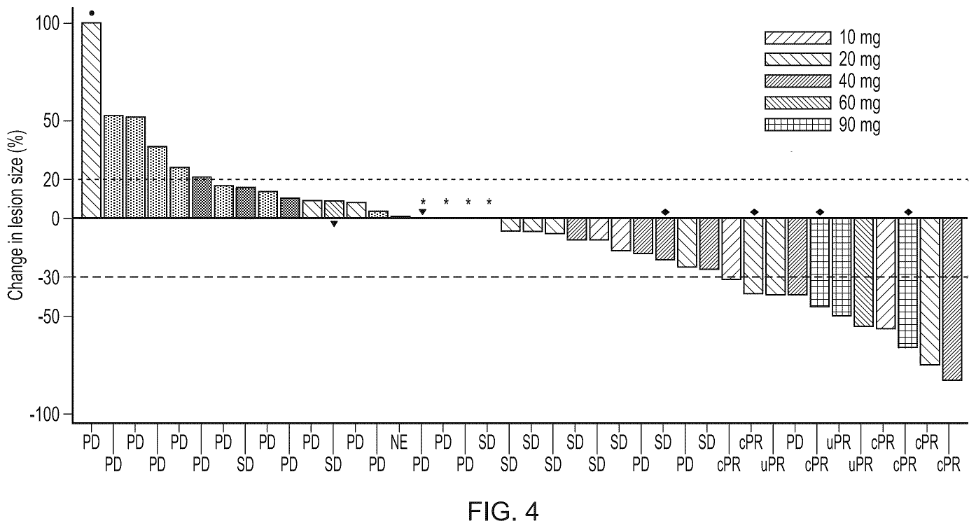

FIGS. 4 to 7 show the response (Best % change in target lesions size by dose) across:
Doses (FIG. 4)
Tumour Types (FIG. 5)
Mutation types (FIG. 6) and
Independent of prior treatment with a PARP inhibitor (FIG. 7).

In each figure, the symbols have the following meanings:

| | |
|---|---|
| ♦ | GCIG CA125 response |
| ▾ | PCWG3 PSA₅₀ response |
| * | Patients with 0% change from baseline |
| ● | Percent change >100 was cut at 100 |

ctDNA Responses

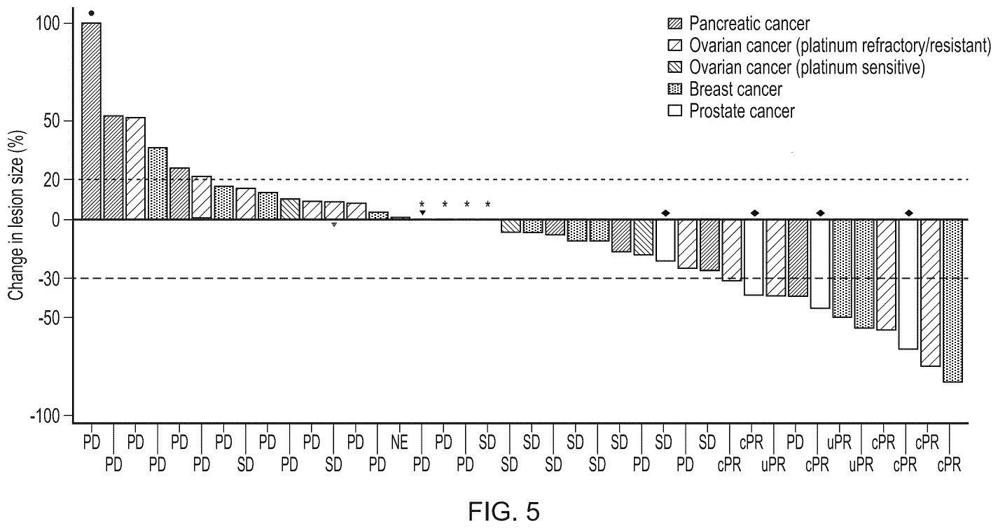

FIG. 8 shows the results of ctDNA measurements on 13 patients.

8 out of 13 patients showed a ctDNA decrease (mean variant allele frequency (mVAF) of all somatic variants detected with at least 0.3% allele frequency; % change was calculated from baseline to C2D1 (Guardant Health OMNI at screening and GH360 thereafter including 5/5 prostate cancer patients

In patients with no measurable disease the PCWG3 PSA₅₀ response (confirmed and unconfirmed) only is represented, using the following abbreviations:
cPR, confirmed Partial Response;
SD, stable disease;
PD, progressive disease;
PSAR, PCWG3 PSA₅₀ response;
PSA⬇, PSA decrease 0-49%

### Individual Patient Results

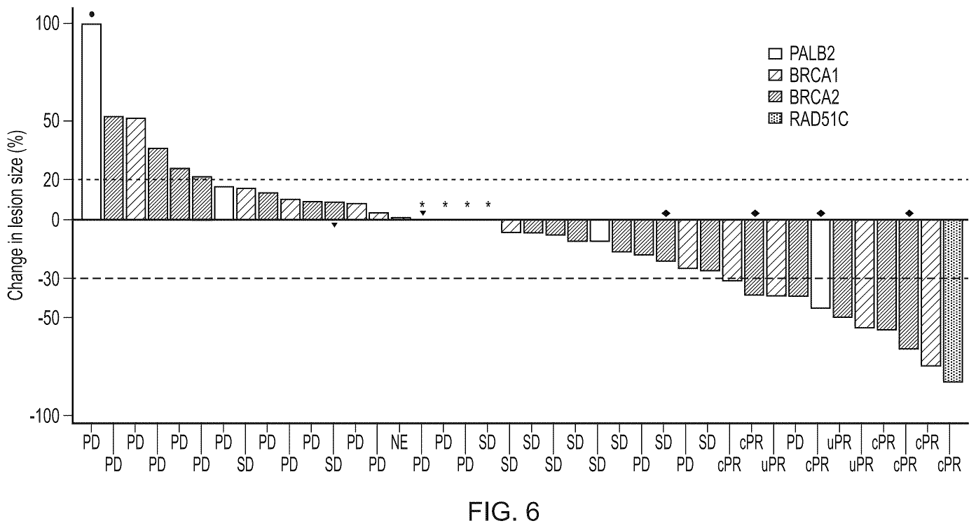

Case 1—66-year-old man with a BRCA2m prostate cancer (AZD5305 20 mg) The patient had been treated with 3 prior lines of therapy: ADT; docetaxel; abiraterone FIG. 9 shows the PSA₅₀ (♦) and ctDNA (▪) responses. A PSA₅₀ partial response (−38.6%) was observed using RECIST, which was confirmed at second scan, and a −58% PCWG3 PSA₅₀ response was observed at C3D1.

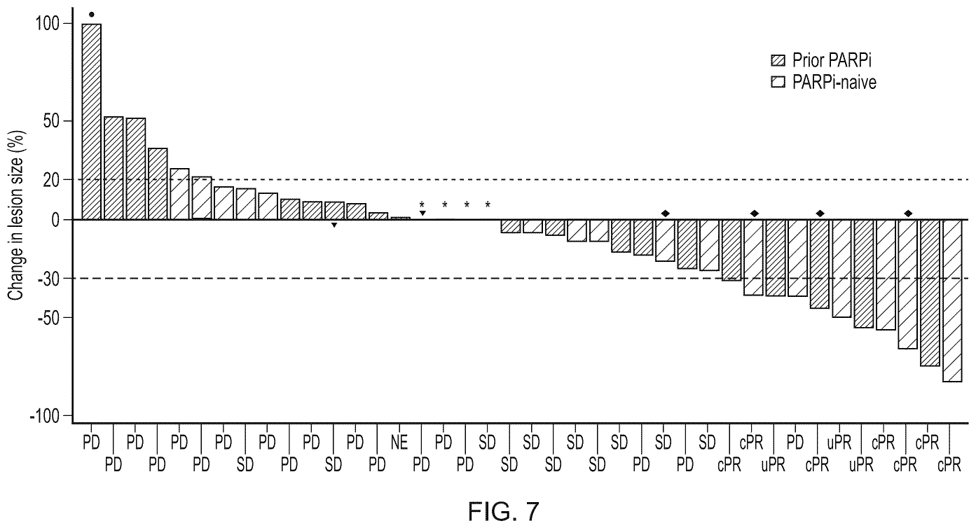

FIG. 10A is the baseline scan (28 Jun. 2021) and FIG. 10B is the second scan (22 Oct. 2021)—the white arrows in FIG. 10A indicate three tumours.

### Cases 2 to 5

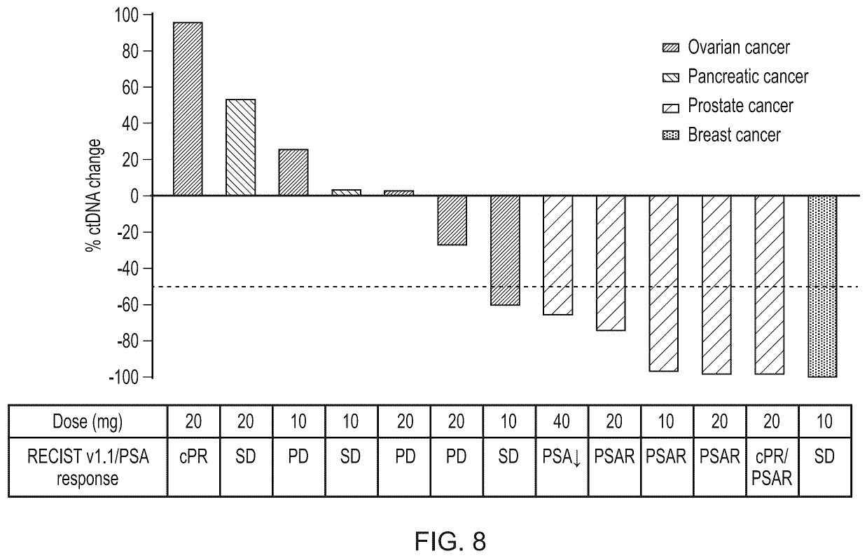

FIGS. 11A to 11D show the PSA₅₀ (♦) and ctDNA (▪) responses of the following 4 prostate cancer patients:
Case #2:56 y/o; BRCA2m; AZD5305 10 mg; 2 prior lines of therapy ADT/abiraterone, and docetaxel
Case #3:55 y/o; BRCA2m; AZD5305 20 mgl; 3 prior lines of therapy: bicalutamide, ADT, abiraterone, and docetaxel
Case #4:60 y/o; BRCA2m; AZD5305 20 mg; 3 prior lines of therapy: docetaxel, enzalutamide, and cabazitaxel
Case #5:60 y/o; BRCA2m; AZD5305 40 mg; 3 prior lines of therapy: bicalutamide, docetaxel, and enzalutamide

These results provide preliminary evidence of early ctDNA responses and association with clinical responses.

Case 6-43 year old woman with gRAD51Cm HR+ breast cancer (AZD5305 40 mg) The patient had been treated with 6 prior lines of therapy:
doxorubicin/cyclophosphamide/paclitaxel; tamoxifen; letrozole/palbociclib;
fulvestrant/Palbociclib; capecitabine/tesataxel; and everolimus/exemestane.

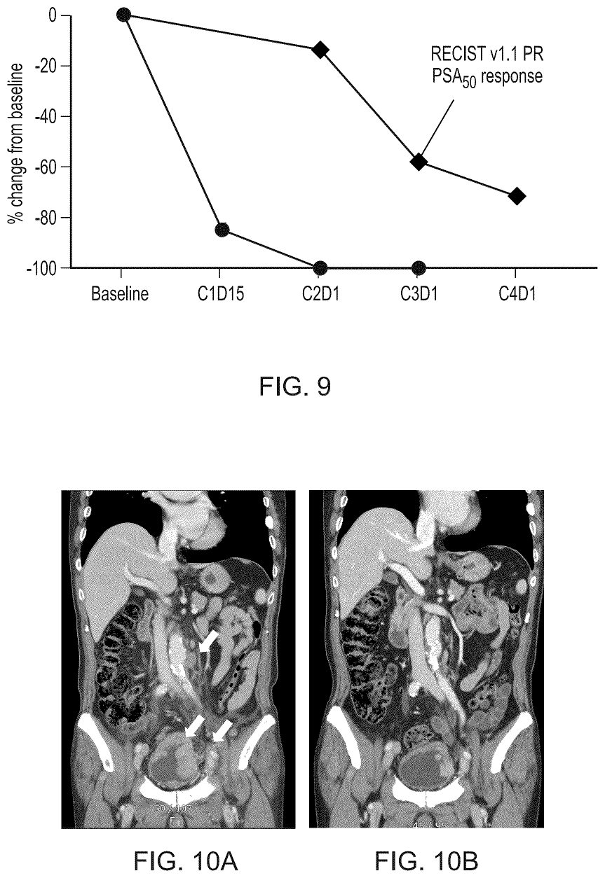

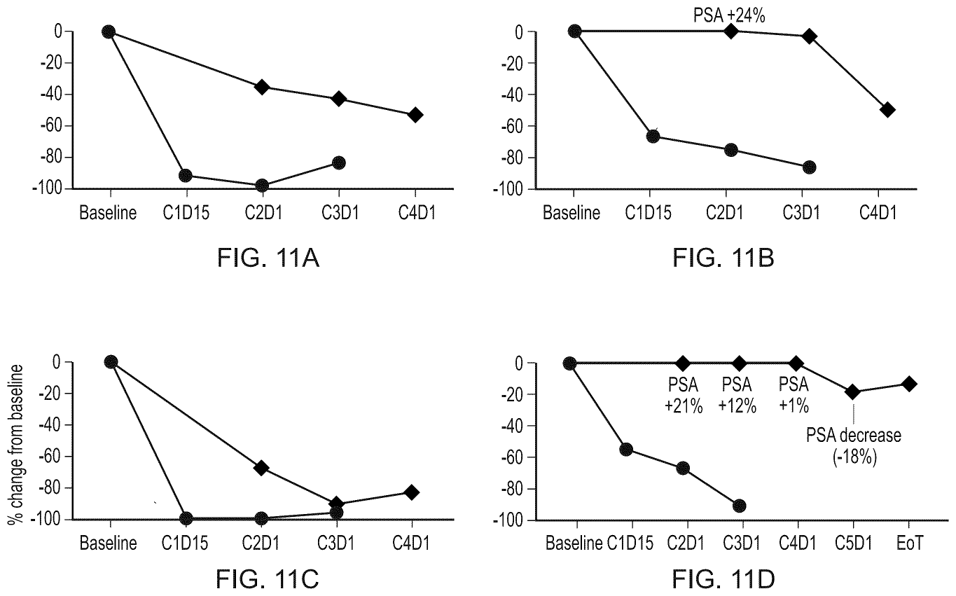

FIG. 12 shows the CA15.3 (U/mL) response, and FIG. 13 shows patient scans. FIG. 13A shows the baseline scans on 11 Nov. 2021, FIG. 13B shows the scans on 18 Jan. 2022, and FIG. 13C shows the scans on 15 Mar. 2022.

A RECIST v1.1 partial response was noted (−83%), which was confirmed at second scan (−86.8%).

The white arrow in FIG. 13A indicates a tumour.

Case 7-53 Year Old Woman with BRCA1m Platinum Resistant Ovarian Cancer (AZD5305 20 mg)

The patient had been treated with 3 prior lines of therapy: carboplatin/paclitaxel, LPD/carboplatin followed by olaparib maintenance (given for 1.6 months, but discontinued for nausea), carboplatin/gemcitabine.

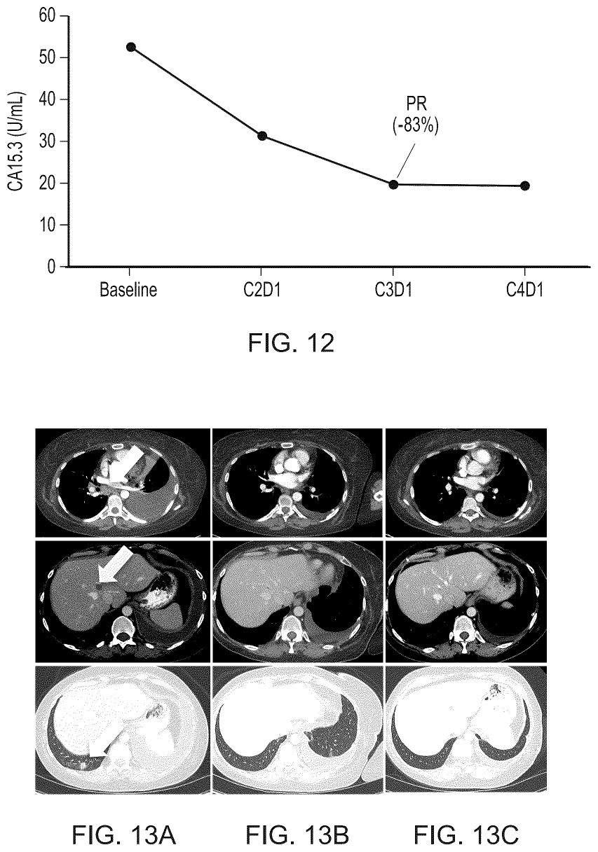

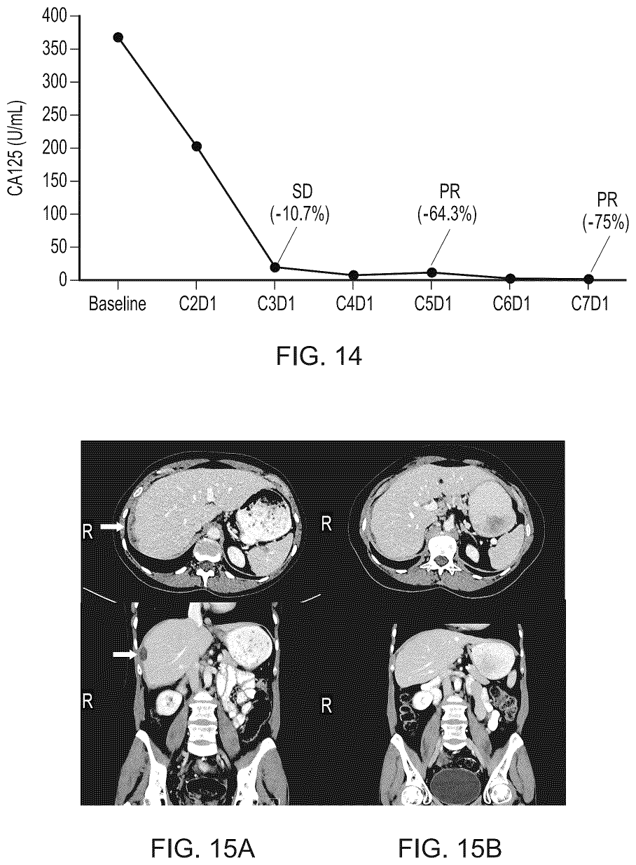

FIG. 14 shows the CA125 (U/mL) response and FIG. 14 shows patient scans. FIG. 15A shows the baseline scans on 18 Jan. 2021, and FIG. 15B shows the third scan on 29 Jul. 2021. A RECIST v1.1 partial response (−75.0%) was confirmed at the third scan. The white arrows in FIG. 15A indicates a tumour.

### REFERENCES

A number of publications are cited above in order to more fully describe and disclose the invention and the state of the art to which the invention pertains. Full citations for these references are provided below. The entirety of each of these references is incorporated herein.

| | Reference | Doi |
|---|---|---|
| Bai 2015 | Bai P. Mol Cell 2015; 58: 947-958 | 10.1016/j.molcel.2015.01.034 |
| Chappuis and | Chappuis P O and Foulkes W D, | 10.1007/978-1-4757-3587-1_2 |
| Foulkes 2002 | Cancer Treat Res, 107, 29-59 | |
| | (2002) | |
| Eisenhauer | Eisenhauer E A, et al., European | 10.1016/j.ejca.2008.10.026 |
| 2009 | Journal of Cancer, 45, 228-247 | |
| | (2009) | |
| Hughes-Davies | Hughes-Davies, et al., Cell, 115, | 10.1016/s0092-8674(03)00930-9 |
| 2003 | 523-535 (2003) | |
| Illuzzi 2022 | Illuzzi G, et al., Clin Cancer Res | 10.1158/1078-0432.CCR-22-0301 |
| | 2022; 28: 4724-4736 | |
| Janatová 2003 | Janatová M, et al., Neoplasma, | PMID: 12937835 |
| | 50(4), 246-50 (2003) | |
| Jancarkova | Jancárková, N, Ceska Gynekol., | PMID: 12708108 |
| 2003 | 68(1), 11-6 (2003) | |
| Jasin 2002 | Jasin M., Oncogene, 21(58), 8981- | 10.1038/sj.onc. 1206176 |
| | 93 (2002) | |
| Johannes 2021 | Johannes, J W, et al., J Med Chem | 10.1021/acs.jmedchem.1c01012 |
| | 2021, 64, 14498-14512 | |
| Khanna and | Khanna K K and Jackson S P, Nat. | 10.1038/85798 |
| Jackson 2001 | Genet. 27(3): 247-254 (2001) | |
| Neuhausen and | Neuhausen S L and Ostrander E A, | 10.1089/gte.1997.1.75 |
| Ostrander 1992 | Genet. Test, 1, 75-83 (1992) | |
| O'Connor 2015 | O'Connor M J, Mol Cell (2015) | 10.1016/j.molcel.2015.10.040 |
| | 60(4): 547-560 | |
| Radice 2002 | Radice, P. J., Exp Clin Cancer | PMID: 12585647 |
| | Res., 21(3 Suppl), 9-12 (2002) | |
| Rostoker 2007 | Rostoker G, et al., J Nephrol. | PMID: 17918143 |
| | 2007; 20(5): 576-585 | |
| Scher 2016 | Scher H I, et al., J Clin Oncol. | 10.1200/JCO.2015.64.2702 |
| | 2016; 34(12): 1402-1418 | |
| Tutt 2002 | Tutt, et al., Trends Mol Med., | 10.1016/s1471-4914(02)02434-6 |
| | 8(12), 571-576, (2002) | |
| Turner 2004 | Turner N, et al., Nat Rev Cancer, | 10.1038/nrc1457 |
| | 2004; 4: 814-819 | |
| Wood 2001 | Wood, et al., Science, 291, 1284- | 10.1126/science. 1056154 |
| | 1289 (2001) | |

1. A method of treatment or prophylaxis of diseases or conditions in which inhibition of PARP1 is beneficial, comprising administering to the subject AZD5305 in a daily dose of 10 to 140 mg, wherein AZD5305 may be in the form of a pharmaceutically acceptable salt.

2. The method according to claim 1, wherein the disease or condition in which inhibition of PARP1 is beneficial is cancer.

3. The method according to claim 2, wherein the cancer is breast, ovary, pancreas, prostate, hematological, gastrointestinal such as gastric and colorectal, or lung cancer.

4. The method according to any one of claims 1 to 3, wherein AZD5305 is administered in a dose of up to 20 mg, 30 mg, 40 mg, 50 mg, 60 mg, 70 mg, 80 mg, 90 mg, 100 mg, 110 mg, 120 mg, 130 mg or 140 mg per day.

5. The method according to any one of claims 1 to 4, wherein AZD5305 is administered in a dose of at least 10 mg, 20 mg, 30 mg, 40 mg, 50 mg, 60 mg, 70 mg, 80 mg, 90 mg, 100 mg, 110 mg, 120 mg or 130 mg per day.

6. The method according to any one of claims 1 to 3, wherein AZD5305 is administered in a dose of 10 mg, 20 mg, 30 mg, 40 mg, 50 mg, 60 mg, 70 mg, 80 mg, 90 mg, 100 mg, 110 mg, 120 mg, 130 mg or 140 mg per day.

7. The method according to any one of claims 1 to 6, wherein AZD5305 is administered once daily.

8. The method according to any one of claims 1 to 7, wherein AZD5305 is administered for 28 days in a 28-day treatment cycle.

9. The method according to any one of claims 1 to 8, wherein AZD5305 is administered orally.

10. The method according to claim 9, wherein AZD5305 is in tablet dosage form.

11. AZD5305, or a pharmaceutically acceptable salt thereof, for use in the treatment or prophylaxis of diseases or conditions in which inhibition of PARP1 is beneficial in a subject, wherein said treatment comprises the administration of said AZD5305 in a daily dose of 10 to 140 mg to said subject.

12. AZD5305, or a pharmaceutically acceptable salt thereof, for use according to claim 11, wherein the disease or condition in which inhibition of PARP1 is beneficial is cancer.

13. AZD5305, or a pharmaceutically acceptable salt thereof, for use according to claim 12, wherein the cancer is breast, ovary, pancreas, prostate, hematological, gastrointestinal such as gastric and colorectal, or lung cancer.

14. AZD5305, or a pharmaceutically acceptable salt thereof, for use according to any one of claims 11 to 13, wherein AZD5305 is administered in a dose of up to 20 mg, 30 mg, 40 mg, 50 mg, 60 mg, 70 mg, 80 mg, 90 mg, 100 mg, 110 mg, 120 mg, 130 mg or 140 mg per day.

15. AZD5305, or a pharmaceutically acceptable salt thereof, for use according to any one of claims 11 to 14, wherein AZD5305 is administered in a dose of at least 10 mg, 20 mg, 30 mg, 40 mg, 50 mg, 60 mg, 70 mg, 80 mg, 90 mg, 100 mg, 110 mg, 120 mg or 130 mg per day.

16. AZD5305, or a pharmaceutically acceptable salt thereof, for use according to any one of claims 11 to 13, wherein AZD5305 is administered in a dose of 10 mg, 20 mg, 30 mg, 40 mg, 50 mg, 60 mg, 70 mg, 80 mg, 90 mg, 100 mg, 110 mg, 120 mg, 130 mg or 140 mg per day.

17. AZD5305, or a pharmaceutically acceptable salt thereof, for use according to any one of claims 11 to 16, wherein AZD5305 is administered once daily.

18. AZD5305, or a pharmaceutically acceptable salt thereof, for use according to any one of claims 11 to 17, wherein AZD5305 is administered for 28 days in a 28-day treatment cycle.

19. AZD5305, or a pharmaceutically acceptable salt thereof, for use according to any one of claims 11 to 18, wherein AZD5305 is administered orally.

20. AZD5305, or a pharmaceutically acceptable salt thereof, for use according to any one of claims 11 to 19, wherein AZD5305 is in tablet dosage form.

21. The use of AZD5305, or a pharmaceutically acceptable salt thereof, in the manufacture of a medicament for use in the treatment of or prophylaxis of diseases or conditions in which inhibition of PARP1 is beneficial, wherein said treatment administering to the subject AZD5305 in a daily dose of 10 to 140 mg.

22. The use of AZD5305, or a pharmaceutically acceptable salt thereof, according to claim 21, wherein the disease or condition in which inhibition of PARP1 is beneficial is cancer.

23. The use of AZD5305, or a pharmaceutically acceptable salt thereof, according to claim 22, wherein the cancer is breast, ovary, pancreas, prostate, hematological, gastrointestinal such as gastric and colorectal, or lung cancer.

24. The use of AZD5305, or a pharmaceutically acceptable salt thereof, according to any one of claims 21 to 23, wherein AZD5305 is administered in a dose of up to 20 mg, 30 mg, 40 mg, 50 mg, 60 mg, 70 mg, 80 mg, 90 mg, 100 mg, 110 mg, 120 mg, 130 mg or 140 mg per day.

25. The use of AZD5305, or a pharmaceutically acceptable salt thereof, according to any one of claims 21 to 24, wherein AZD5305 is administered in a dose of at least 10 mg, 20 mg, 30 mg, 40 mg, 50 mg, 60 mg, 70 mg, 80 mg, 90 mg, 100 mg, 110 mg, 120 mg or 130 mg per day.

26. The use of AZD5305, or a pharmaceutically acceptable salt thereof, according to any one of claims 21 to 23, wherein AZD5305 is administered in a dose of 10 mg, 20 mg, 30 mg, 40 mg, 50 mg, 60 mg, 70 mg, 80 mg, 90 mg, 100 mg, 110 mg, 120 mg, 130 mg or 140 mg per day.

27. The use of AZD5305, or a pharmaceutically acceptable salt thereof, according to any one of claims 21 to 26, wherein AZD5305 is administered once daily.

28. The use of AZD5305, or a pharmaceutically acceptable salt thereof, according to any one of claims 21 to 27, wherein AZD5305 is administered for 28 days in a 28-day treatment cycle.

29. The use of AZD5305, or a pharmaceutically acceptable salt thereof, according to any one of claims 21 to 28, wherein AZD5305 is administered orally.

30. The use of AZD5305, or a pharmaceutically acceptable salt thereof, according to claim 29, wherein AZD5305 is in tablet dosage form.
